(19) United States
(12) Patent Application Publication
(10) Pub. No.: US 2022/0233304 A1
(43) Pub. Date: Jul. 28, 2022

(54) METHOD OF SURGICALLY IMPLANTING AN INTRAOCULAR LENS (IOL) USING A CAPSULAR PROSTHESIS TO SUPPORT POSTERIOR CHAMBER FIXATION

(71) Applicant: Mark Michael Femandez, Norfolk, VA (US)

(72) Inventor: Mark Michael Femandez, Norfolk, VA (US)

(21) Appl. No.: 17/512,542

(22) Filed: Oct. 27, 2021

Related U.S. Application Data
(63) Continuation-in-part of application No. 17/156,694, filed on Jan. 25, 2021, now abandoned.

(51) Int. Cl. A61F 2/16

(52) U.S. Cl. CPC A61F 2/1662; A61F 2002/16901

(57) ABSTRACT

A capsular prosthesis is disclosed that is implanted to support posterior chamber placement of IOLs. The capsular prosthesis can be implanted to replace the capsule in situations where the patient's natural capsule has been rendered incapable of providing the structural support necessary to maintain proper centration of an IOL implanted therein. The prosthesis is surgically implanted into the eye by inserting the prosthesis into the eye through a primary incision. The prosthesis is secured to the sclera of the eye with at least two transscleral sutures to establish at least three points of contact between the sclera and the at least three apertures of the prosthesis. The at least two transscleral sutures support the sheet within a desired plane located within the posterior chamber, the plane containing a predetermined surgical axis passing through the center aperture. The IOL is inserted through the primary incision and optically captured on the prosthesis.

### CROSS-REFERENCE TO RELATED APPLICATIONS

This application is related to US Pat. App. No. titled “A CAPSULAR PROSTHESIS FOR POSTERIOR CHAMBER INTRAOCULAR LENS (IOL) FIXATION,” and which is hereby incorporated herein in its entirety by this reference.

### FIELD OF THE INVENTION

The invention relates to intraocular lens (IOL) implantation, and more particularly to techniques for the surgical implantation of such lenses using a prosthesis in situations where capsular support is inadequate or non-existent.

### BACKGROUND OF THE INVENTION

Cataract surgery is one of the most frequently and successfully performed surgeries performed on the human eye. The American Society of Cataracts and Refractive Surgery (ASCRS) estimates that 3 million Americans undergo cataract surgery each year, with an overall success rate of 98 percent or higher. A cataract is simply defined by clouding or discoloration of the crystalline lens that makes it difficult to focus light onto the retina 30. When this occurs, a cataract surgeon removes the crystalline lens and replaces it with an artificial intraocular lens (i.e. IOL) that is able to properly focus light once again onto the retina (30, FIG. 1A) correctly.

FIG. 1A is a simplified illustration of the anatomy of the human eye 10. The crystalline lens 26 of the eye 10 has a nucleus 31 encased by a membranous bag-like structure called a capsule 24, which is divided into the posterior 28 and anterior 34 capsules. The capsule 24 lies within the anterior segment 19 of the eye 10, which is the front third of the eye 10 located in front of the vitreous humor 11, and includes the cornea 14, iris 12, ciliary body 21, and crystalline lens 26. The crystalline lens 26 is generally located posterior to the iris 12. The anterior chamber 16 is the space between the iris 12 and the cornea 14. The crystalline lens 26 is suspended in place within the posterior chamber 17 by fine suspensory fibers called zonules 22 originating from the ciliary body 21.

The crystalline lens 26 is generally aligned with the optical axis A-A′ 55. It extends through the geometric center of the cornea 16 to the geometric center of the retina 30, approximately halfway between the optic nerve 31 and the fovea 32. The optical axis A-A′ 55 is defined by the geometric centers of cornea 16, pupil 20, and retina 30. However, the visual axis B-B′ 59 is the actual axis through which the human eye looks, which runs from a person's point of fixation to the fovea 32. The angle α 58 between the optical A-A′ 55 and visual 59 axes is about 5.2°.

A number of techniques are available to remove cataracts, and the one ultimately employed by the surgeon is dependent upon factors such as how advanced the cataracts are and the health of the patient's eyes generally. Phacoemulsification is the most commonly employed and desirable technique. The surgeon first tears a circular hole (i.e. capsulorhexis) (See 40, FIG. 1B) in the anterior capsule 34 to access the cataract. The crystalline lens 26 is loosened from the capsule 24 by injecting saline solution between the capsule 24 and the cataractous lens 26 material. The lens 26 material internal to the capsule 24 is liquified and aspirated from the eye using a phacoemulsification device (e.g. a metal cannula that vibrates at ultrasonic frequency). The device breaks up the cloudy cataract into tiny fragments that are removed from the eye 10 using suction.

As long as the capsule 24 remains largely intact other than the hole 40 (i.e. capsulorhexis) through which the affected crystalline lens 26 is removed, an IOL 70, 80 (such as the ones illustrated in FIGS. 2A, B) is inserted through the incision and capsulorhexis 40 and is implanted within the capsular bag 24 in place of the removed crystalline lens 26. Capsular placement or implantation is the optimal location anatomically for an IOL intended to replace the removed cataract. It provides optimal stability and permits the optic 72, 82 of the IOL to be located closest to the nodal point of the original nucleus 31 of the crystalline lens 26, through which the optical axis A-A′ 55 of the eye passes and which is substantially aligned with their centroid 76, 86.

There are many types of intraocular lenses 70, 80 currently available, and are typically either a single-piece design 70, or a three-piece design 80. The choice of IOL is at least partially dictated by the therapeutic purpose to be served, as well as its suitability to the location within the eye where the IOL ultimately will be placed. IOL's all have an optic 72, 82 to focus the light on the retina 30 in lieu of the removed crystalline lens 26, and arms (or haptics) 74, 84 that provide a reactive force to help hold and center the optic 72, 82 in a fixed position, with centroid 76, 86 substantially aligned with a desired axis of the eye (e.g. either the optical axis A-A′ 55 or visual axis B-B′ 59) at center 76, 86 as illustrated in FIG. 1B. Centration of the optic 72, 82 is important to obtaining desired visual acuity. Most lenses have been designed with their centroid 76, 86 to be aligned with the center of the pupil 20 (and thus the optical axis A-A′ 55) even though the visual axis 59 does not pass through this point. For spheric and aspheric lenses, this does not affect the visual acuity significantly. For newer lens technologies such as multifocal lenses, it may be more desirable to center the centroid 76, 86 of its optic 72, 82 with the visual axis B-B′ 59 for optimal visual acuity.

Single piece IOL's 74, FIG. 2A are usually made entirely (both optic 72 and haptics 74) from hydrophobic or hydrophilic acrylic. As a result, single piece IOLs have haptics 74a, b that are soft and broad. They are often preferred for placement in reasonably intact capsules 28, 34. Single-piece toric lenses are designed to correct for a patient's astigmatism. The optics 82 of three-piece IOLs 80, FIG. 2B are made from acrylic, silicone, or another suitable elastomer, and have haptics 84a, b that are made separately from the optic 82 and attached thereto. The haptics 84a, b are typically made of a different material such as polymethyl-methacrylate (PMMA) or polypropylene. Both are suitable for placement of an optic within the capsule as illustrated in FIG. 1B.

For many reasons, the capsule 24 is not always left sufficiently intact to support implantation of the IOL 70, 80 within the capsule 24 as shown in FIG. 1B. For example, a second surgical technique called extracapsular cataract surgery is sometimes employed in situations where the condition of the eye prevents the use of the more desirable phacoemulsification. One such situation is when the cataracts are more advanced, which renders them too dense for phacoemulsification. Extracapsular surgery requires a larger incision in the cornea 14, which requires sutures for proper healing of that larger incision. In addition, it is not uncommon that during cataract surgery or long after, a number of complications can occur that can make it impossible to securely place an artificial IOL lens 70, 80 within the capsule. For example, the posterior capsule 28 can rupture during surgery such that a large hole (in addition to the surgically created capsulorhexis 40) in the capsular bag 24 precludes placing an IOL within it.

In cases where capsular placement of an IOL is not possible, a three-piece IOL 70 can be placed within the ciliary sulcus 18. FIG. 1C illustrates such a placement. The haptics 84a, b can be seen located in the sulcus 18, and the optic 82 is located anterior to the anterior capsule 34 and the capsulorhexis 40 made therein to remove the cataract. Unfortunately, for this type of placement, the long term centration of the optic 82 of the IOL 80 to the optical axis A-A′ 55 can become compromised. Moreover, the haptics 84a, b can migrate and rub against the iris 12, causing irritation thereto and depigmentation thereof. Patients often require a second procedure months or years after the first surgery to suture the lens 80 to the iris 12 or sclera 36 so that it does not fall into the posterior chamber (not shown), and to recenter the lens 80 to the optical axis A-A′ 55 so that it properly focuses light onto the retina 30.

If the anterior capsule 34 is reasonably intact, and the zonules 22 are able to still support the anterior capsule, an alternative technique for ciliary sulcus 18 placement (not pictured) can be used called reverse optic capture. In this technique, a three piece IOL (80, FIG. 2B) can be placed such that the haptics 84a, b are anterior to the anterior capsule 34, FIG. 1C and the optic 82 of the IOL 80 is then prolapsed posteriorly so that the optic 82 is forced through an intact capsulorhexis 40 in the anterior capsule 34 and is held in place thereby. Placement within the ciliary sulcus 18 via reverse optic capture is a more stable technique by which to achieve an IOL 80 with proper centration with respect to the optical axis A-A′ 55 (as defined by the iris 12 and the pupillary border 44) notwithstanding a damaged posterior capsule 24, than is the simpler sulcus placement of FIG. 1C.

Another technique used for anterior segment 19 placement of an IOL 90 is to suture a three piece IOL to the iris 12. Although a relatively good technique, it is technically difficult with a lengthy procedure that includes a steep learning curve. In addition to being difficult to perform, it is not unusual for the lens to chafe the iris 12, causing inflammation or for the lens to dislocate.

In some situations, the entire capsule 24 complex (anterior 34 and posterior 28 capsule) is damaged and/or removed (see FIG. 1D), or the zonules 22 are damaged so extensively that ciliary sulcus 18 placement of the IOL 80, with or without reverse optic capture simply cannot be performed. Thus, the next available mode of IOL (90, FIG. 1D) placement will typically be within the anterior chamber 16 of the eye 10. Those of skill in the art will appreciate that FIG. 1D is intended to illustrate anterior IOL 90 placement in general, and not the fine details of any specific such anterior chamber lens design or technique. Currently, the most common way to address this complication is to make an even larger incision by which to place an anterior chamber lens (ACIOL) anterior to the iris 12. While this technique is relatively simple, the large incision slows healing and the technique is more likely to cause failure of the cornea 14, requiring corneal transplantation later in life.

In another known technique for anterior chamber 16 placement, an IOL 90 can be sutured directly to the white part of the eye (i.e. sclera 36). While this technique of anterior chamber placement does not damage the cornea 14, it is often performed using a larger rigid lens which requires a commensurately larger incision. Because almost all lenses used for this technique have only two haptics, many of which are designed with varying angulation, only two effective points of contact exist between the IOL and the sclera 36, making it easy for the surgeon to inadvertently place the lens 90 in a way that it will rotate and rub against the iris 12. This can lead to iris chafe and inflammation within the eye. Finally, because many of the techniques discussed above require suturing the lens to the eye, it renders any efforts to replace those lenses a significant surgery in and of itself.

It would be desirable to avoid IOL placement after cataract surgery anterior to the capsule 24 in situations where the capsule 24 is not able to support placement therein, and particularly to avoid placements within the anterior chamber 16. Placement within the capsule 24 is the natural position for lens placement and avoids the complications that can occur for placements within the anterior chamber 16, and also within the sulcus 18. It would also be desirable to minimize the invasiveness of procedures required to replace previously implanted lenses. It would be further desirable to facilitate a more uniform but flexible technique for lens replacement regardless of the type of IOL used, and to provide more freedom to achieve a desired centration of the optic.

### SUMMARY OF THE INVENTION

A capsular prosthesis of the invention is disclosed that is configured to be implanted to support placement of IOLs in a position that substantially corresponds to the location of the naturally occurring crystalline lens provided by an intact capsule of the human eye prior to its removal. The capsular prosthesis can be implanted to essentially replace the capsule in situations where the patient's natural capsule has been rendered incapable of providing the structural support necessary to maintain proper centration of an IOL implanted therein. A method of implanting the prosthesis is further disclosed.

In one aspect of the invention, a method of surgically implanting an intraocular lens (IOL) into an eye using a capsular prosthesis to support posterior chamber fixation includes providing a capsular prosthesis comprising a sheet of substantially biocompatible and/or bioinert material. The sheet further includes an anterior and posterior face separated by a thickness, three or more vertices, each one of the vertices being uniquely associated with a suture aperture located proximally with its point, and a center aperture located centrally with the vertices and being dimensionally configured to permit supportive optical capture of the IOL without substantial impairment of optic functionality.

The prosthesis is surgically implanted into the eye by inserting the prosthesis into the eye through a primary incision. The prosthesis is secured to the sclera of the eye with at least two transscleral sutures to establish at least three points of contact between the sclera and the at least three apertures of the prosthesis. The at least two transscleral sutures to support the sheet within a desired plane located within the posterior chamber, the plane containing a predetermined surgical axis passing through the center aperture, the plane being approximately perpendicular to, and the center aperture of the sheet being functionally centered with, the predetermined axis of the eye. The IOL is inserted through a primary incision and optically captured on the prosthesis so that a center of the optic is approximately centered with the aperture and the predetermined axis of the eye.

In an embodiment, a first one of the at least two transscleral sutures is secured to a first set of one or more of the at least three suture apertures by looping the first transscleral suture through each of the first set of the suture apertures, and a second one of the at least two transscleral sutures is secured to a second set of one or more of the at least three suture apertures by looping the second transscleral suture through each of the second set of the suture apertures.

In an embodiment, the sheet of the prosthesis is substantially rectangular, and the first set of the suture apertures includes two of the suture apertures each located proximally with a different one of two vertices located at a first end of the sheet. The second set of the suture apertures includes two of the suture apertures each located proximally to a different of two vertices at a second end of the sheet.

In a further embodiment, the sheet of the prosthesis is substantially triangular in geometry, and the first set of the suture apertures includes an aperture located at the apex of the triangular sheet. The second set of the suture apertures includes two of the suture apertures each located proximally with a different one of the two vertices defining the base of the triangular sheet.

In a still further embodiment, each of the first and second looped transscleral sutures has two paired ends, and each one of the paired ends of the first looped transscleral suture are secured to the sclera of the eye through a sclerotomy made proximally with a first predetermined point along the predetermined surgical axis. Each one of the paired ends of the second looped transscleral suture are secured to the sclera of the eye through a sclerotomy made proximally with a second predetermined point located along the predetermined surgical axis and 180 degrees from the first predetermined point.

In a further embodiment, the first and second predetermined points are about 4 mm posterior to the surgical limbus of the eye.

In an embodiment, the sclerotomy points identified for each of the paired ends of the first and second transscleral sutures are on opposite sides of the predetermined surgical axis approximately 3 mm from the first and second predetermined points respectively.

In an embodiment, the sclerotomy for each of the paired ends of the first and second looped transscleral sutures is made on opposite sides of the predetermined surgical axis, approximately 3 mm from the first and second predetermined points respectively.

In another embodiment, the primary incision is made at a first predetermined incision point along the predetermined surgical axis.

In a still further embodiment, the primary incision is made at a first incision point along an axis that is approximately perpendicular to the predetermined surgical axis.

In another aspect of the invention, the first and second looped transscleral sutures are loaded through the first and second sets of apertures respectively prior to surgery, each of the paired ends being coupled to a surgical needle.

In an embodiment, securing the prosthesis to the sclera further includes, for each of the paired ends of the first transscleral suture, making a sclerotomy from outside of the eye substantially at the identified sclerotomy point using a hollow needle until a proximal end of the hollow needle becomes visible behind the pupil of the eye, inserting the surgical needle into the eye through the primary incision. The inserted needle is docked into the proximal end of the hollow needle and loaded the inserted needle until the inserted needle emerges outside of a distal end of the hollow needle remaining outside of the eye.

In an embodiment, securing the prosthesis to the sclera further includes, for each of the paired ends of the second transscleral suture, making a sclerotomy from outside of the eye substantially at the identified sclerotomy point using a hollow needle until a proximal end of the hollow needle becomes visible behind the pupil of the eye, inserting the surgical needle into the eye through the primary incision. The inserted needle is docked into the proximal end of the hollow needle and loaded the inserted needle until the inserted needle emerges outside of a distal end of the hollow needle remaining outside of the eye.

In another embodiment, the sclerotomy points that are identified for each of the paired ends of the first and second transscleral sutures are on opposite sides of the predetermined surgical axis approximately 3 mm from the first and second predetermined points respectively.

In yet another embodiment, the primary incision is made at a first predetermined incision point along the predetermined surgical axis.

In a further embodiment, the primary incision is made at a first incision point along an axis that is approximately perpendicular to the predetermined surgical axis.

In another embodiment, the first and second looped transscleral sutures are loaded through the first and second sets of apertures respectively prior to surgery, each of the paired ends being coupled to a surgical needle.

In a further embodiment, securing the prosthesis to the sclera further includes: for each of the paired ends of the first transscleral suture, making a sclerotomy from outside of the eye substantially at the identified sclerotomy point using a hollow needle until a proximal end of the hollow needle becomes visible behind the pupil of the eye, inserting the surgical needle into the eye through the primary incision; and docking the inserted needle into the proximal end of the hollow needle and loading the inserted needle until the inserted needle emerges outside of a distal end of the hollow needle remaining outside of the eye.

In still another embodiment, said securing the prosthesis to the sclera further includes: for each of the paired ends of the second transscleral suture, making a sclerotomy from outside of the eye substantially at the identified sclerotomy point using a hollow needle until a proximal end of the hollow needle becomes visible behind the pupil of the eye, inserting the surgical needle of the paired end into the eye through the primary incision; and docking the inserted needle into the proximal end of the hollow needle and loading the inserted needle until the inserted needle emerges outside of a distal end of the hollow needle remaining outside of the eye.

In another embodiment, after removing the needles from the paired ends, both paired ends of the first suture are pulled to pull the prosthesis within the eye through the primary incision.

In another embodiment, the paired ends of both sutures are pulled to suspend the prosthesis within the eye and so that it approximately occupies the desired plane.

In another embodiment, the IOL is pulled into the eye through the primary incision using a standard lens insertion cartridge.

In a still further embodiment, the optic is manipulated with a surgical instrument so that its longitudinal edges are in contact with one of the faces of the prosthesis, so that the optic is substantially centered with the center aperture of the prosthesis, and the haptics of the IOL are captured within vertex features defined by the center aperture to resist further displacement.

In yet another embodiment, the predetermined surgical axis is determined to match the axis of astigmatism of the eye to facilitate easier placement of the IOL, and the IOL is a single-piece toric lens.

In another embodiment, securing the prosthesis to the sclera includes subjecting each of the paired ends of the first and second looped transscleral sutures to heat cautery to make thickened flanges to secure the looped transscleral sutures within the sclera of the eye.

In an embodiment, securing the prosthesis to the sclera further includes tying the paired ends of the first and second looped transscleral sutures to secure the looped transscleral sutures within the sclera of the eye.

### BRIEF DESCRIPTION OF THE DRAWINGS

FIG. 1A is a simplified cross-sectional side view illustration of the anatomy of the human eye;

FIG. 1B is a simplified cross-sectional side view illustration of a human eye from which a cataract has been surgically removed and replaced with an intraocular lens (IOL) that has been implanted in the capsule using techniques known to those of skill in the art;

FIG. 1C is an image of a human eye from which a cataract has been surgically removed and replaced with an intraocular lens (IOL) placed in the sulcus in accordance with techniques known to those of skill in the art;

FIG. 1D is a simplified cross-sectional side view illustration of a human eye from which a cataract has been surgically removed and replaced with an intraocular lens (IOL) that has been surgically implanted into the anterior chamber in accordance with techniques known to those of skill in the art;

FIG. 2A is a plan view of a single piece IOL known to those of skill in the art;

FIG. 2B is a plan and side view of a three-piece IOL known to those of skill in the art;

FIG. 3A is a plan view of a rectangular embodiment of a capsular prosthesis;

FIG. 3B is a side view of the embodiment of the capsular prosthesis of FIG. 3A;

FIG. 4A is an elevated anterior view of the embodiment of the prosthesis of FIGS. 3A and B with a three-piece IOL reverse optically captured thereon;

FIG. 4B is a side view of the embodiment of the prosthesis of FIGS. 3A, B and FIG. 4A with a three-piece IOL reverse optically captured thereon;

FIG. 5 is a plan view of a triangular embodiment of the capsular prosthesis;

FIG. 6A is a is an elevated anterior view of the embodiment of FIG. 5 with a one-piece IOL optically captured thereon;

FIG. 6B is a is an elevated posterior view of the embodiment of FIGS. 5 and 6A with a one-piece IOL optically captured thereon;

FIG. 7 is a plan view of a human eye within which the embodiment of the prosthesis of FIGS. 3A, B and 4A, B (or alternatively the embodiment of FIGS. 5A, 6A, and 6B) has been surgically implanted through an embodiment of method of surgical implantation of the invention;

FIGS. 8A-H each illustrate, through a plan view of the human eye, one of a series of surgical stages of a method of surgical implantation of the invention by which an embodiment of the prosthesis is surgically implanted to achieve the result illustrated in FIG. 7;

FIG. 9A is a plan view of a rectangular embodiment of the capsular prosthetic, having been surgically implanted within the eye in accordance with the surgical implantation procedure of FIGS. 8A-H, supporting a one piece IOL through optic capture;

FIG. 9B is a plan view of a triangular embodiment of the capsular prosthetic, having been surgically implanted within the eye in accordance with the surgical implantation procedure of FIGS. 8A-H, supporting a three piece IOL through reverse optic capture;

FIGS. 10A-D illustrate surgical steps that may be substituted for steps illustrated in FIGS. 8A-H for an alternate method of the invention for implanting the prosthesis;

FIG. 11A is a diagnostically produced visual representation of a patient's axis of astigmatism;

FIG. 11B is a plan view of an adjusted surgical placement of the embodiment of the prosthesis of the invention of FIGS. 3A, B and 4A, B having a one-piece toric IOL optically captured thereon, to compensate for the angle of astigmatism of FIG. 11A; and

FIG. 12 is an illustration of the triangular prothesis of FIG. 9B having one transscleral suture per suture aperture.

### DETAILED DESCRIPTION

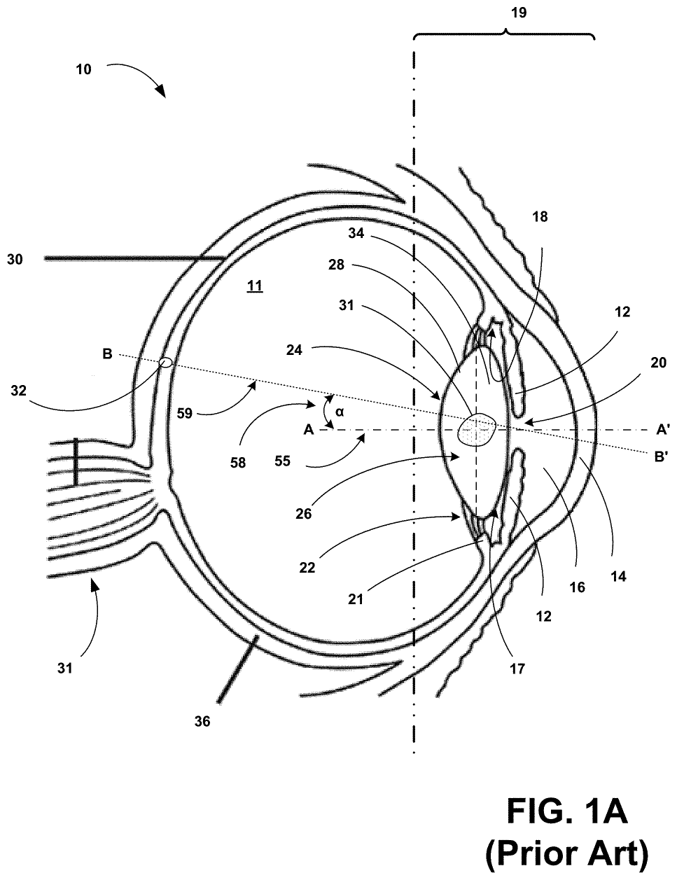

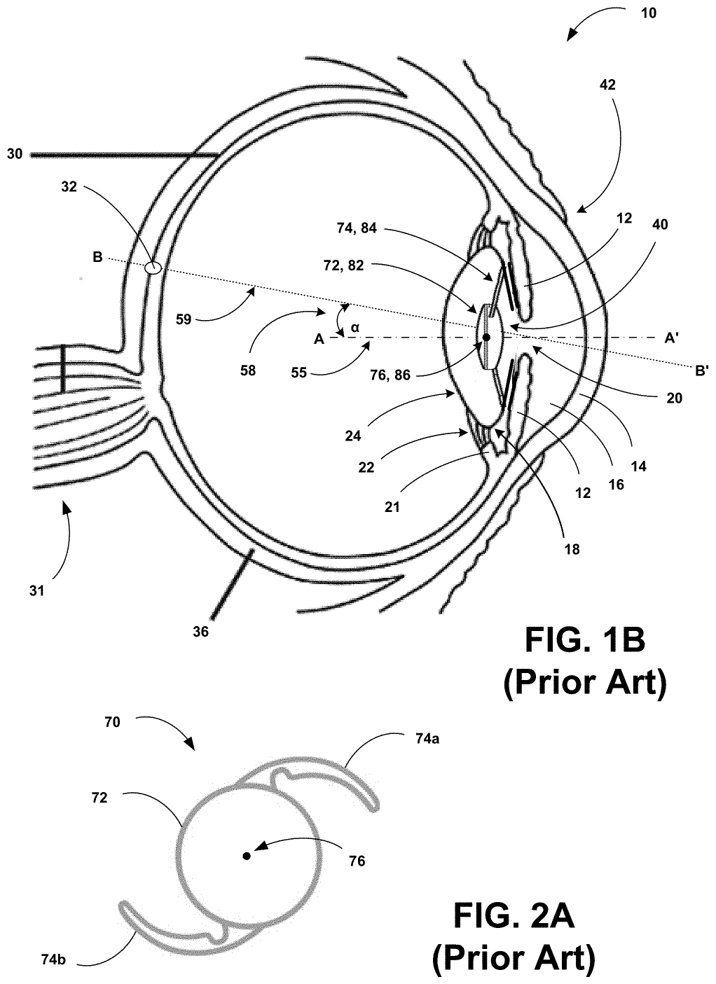

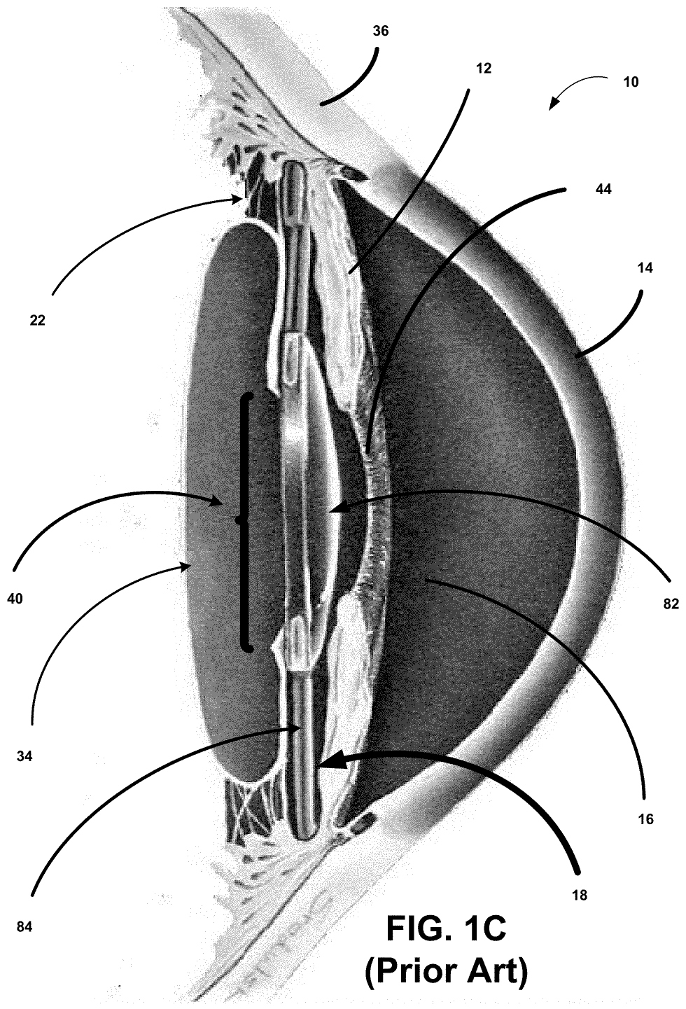

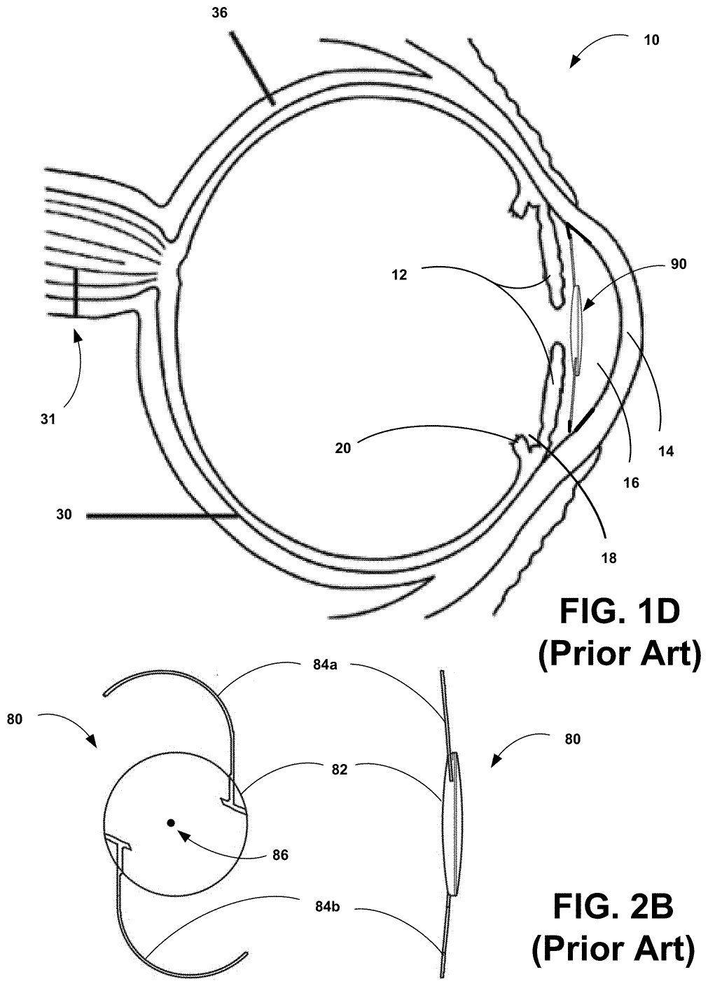

Embodiments of methods for surgically implanting a capsular prosthesis are disclosed. The prosthesis is implanted in accordance with methods of the invention to receive and support commercially available single and three-piece IOL's 70, 80 (FIGS. 2A, 2B) via their haptics 74, 84 (FIGS. 2A, 2B) by way of a prosthetic optic capture to secure the lens 70, 80 (FIGS. 2A, 2B) to the prosthesis and to hold accurate centration with the center 76, 86 of its optic 72, 82 (FIGS. 2A, 2B) with a desired axis of the eye thereby. This eliminates the need to suture the haptics 74, 84 (FIGS. 2A, 2B) themselves to either the sclera 36, FIG. 1A or the iris 12, FIG. 1A as is often required of anterior chamber 16, FIG. 1D and some sulcus 18, FIG. 1C placement techniques in lieu of capsular implantation (FIG. 1D) when it is not practicable. Moreover, the surgical methods of implanting the prosthesis 100, 200 of the invention serve to normalize placement of the various lens designs with their haptics independent of varying materials, lengths, and degrees of angulation. The methods of surgical implantation of prosthesis 100, 200 simplify placement of the lens optic 72, 82 planar to the iris 12 and with substantially optimal centration to a desired axis of the eye 50, such as optical axis A-A′ 55 or visual axis 59, regardless of the design or composition of the IOL used.

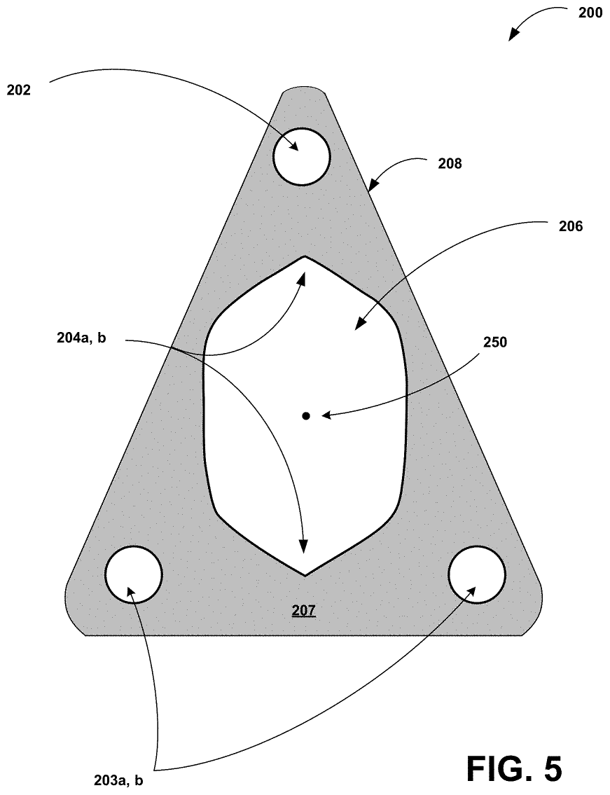

The methods of implantation and features of prosthesis 100, 200 provide a plurality of points of contact greater in number than just the two typically provided by the haptics of an IOL alone. This renders the IOL largely immune from torquing after implantation, as well as eliminating the need for post-operative adjustments of the IOL to achieve optimal centration with the eye's optical A-A′ 55 or visual 59 axis. These points of contact are made by way of at least two looped sutures, one proximal and one distal to the surgeon, which are looped through prosthesis 100, 200 and introduced through the sclera 36. These points are predetermined by the surgeon to achieve a desired surgical axis C-C′ (60, FIGS. 5A, 6A, 8) for placement of the prosthesis that defines a plane that is perpendicular to the desired axis to which the IOL 70, 80 is to be centered. Centration can be achieved by pulling on the paired ends of each of the two sutures before they are surgically fixed within the sclera 36 (e.g. tying them into knots, subjecting each of the paired ends to heat cautery, etc.). This serves to suspend the flexible but resilient prosthesis like a trampoline to support the IOL thereon.

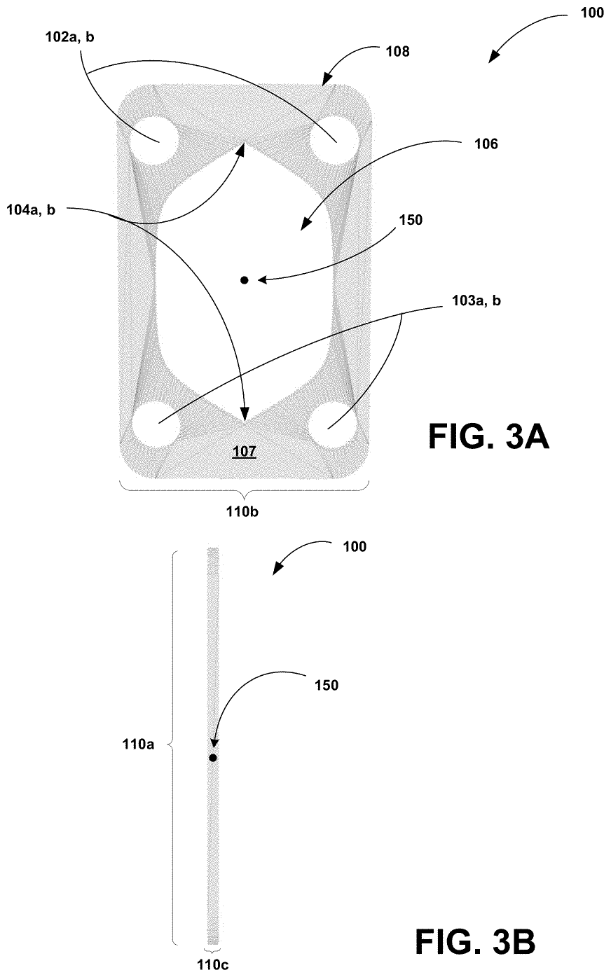

Through the methods of implantation of the invention, the capsular prosthesis 100, 200 is surgically secured within the posterior chamber 17 (in the space normally occupied by the anterior capsule 34). As a result, the prosthesis of the invention (100, 200 of FIGS. 3A, 3B, 4A, 4B, 5, and 6A, B) has been configured to support a three-piece IOL having been secured thereon using reverse optic capture, or a single piece IOL via optic capture. This enables standard IOLs to be placed in substantially the same concentric alignment as that previously provided by the patient's pre-operative capsule for the removed cataract. Surgical implantation of the capsular prosthesis thereby eliminates the need for virtually all of the less than ideal placement techniques of IOLs in the ciliary sulcus 18 or the anterior chamber 16, and particularly in situations where the capsule 24 is not sufficiently sound to support capsular implant of an IOL 70, 80 within the naturally occurring capsule 24.

The prosthesis 100, 200 of the invention essentially replicates sulcus 18 placement of three-piece IOLs 80 with reverse optic capture, in that the center aperture 106, 206 of prosthesis 100, 200 of the invention acts in lieu of an intact capsulorhexis 40 of an anterior capsule when using reverse optic capture for a sulcus placement of an IOL. It can also be used to accomplish optic capture of one-piece IOLs 80 by capturing the optic 82 on the anterior side of the prosthesis and prolapsing the haptics to the posterior side of the prosthesis. The haptics 84 are placed though the center aperture 106 and forward of the anterior capsule 34, and the optic 82 of the three-piece lens 80 is captured against the prosthesis similar to the manner in which it is captured if it were prolapsed through the capsulorhexis of the anterior capsule 34. Alternatively, if a one-piece IOL 70 is used that cannot safely be placed in a reverse optic capture orientation, the haptics 74 can be prolapsed posterior to the prosthesis 100, 200 with the optic 72 being placed anterior to the prosthesis 100, 200.

Existing methods of lens placement and fixation, particularly within the anterior chamber 16, involve fixating the IOL to structures in the eye 50 itself using sutures. Thus, when replacing that IOL when indicated by, for example, a poor refractive outcome, such replacement becomes a major surgical procedure to remove the sutures of the IOL to be replaced, and then suturing in a new one. The prosthesis 100, 200 of the invention facilitates easy lens replacement through a small incision, because the implanted prosthesis 100, 200 itself does not have to be removed to replace the IOL. Replacement simply requires that the existing IOL supported by the prosthetic be removed and replaced with a new lens being supported by the previously implanted prosthetic. Thus, easy fixation of various commercially available IOL designs to the prosthesis 100, 200 of the invention renders IOL removal and replacement simple and less invasive.

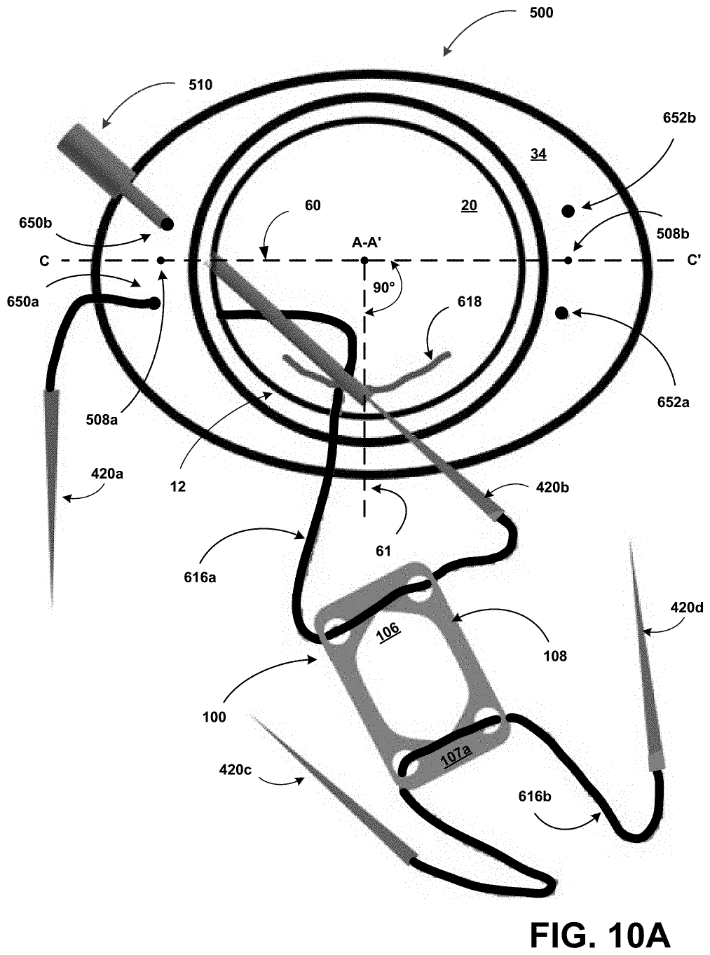

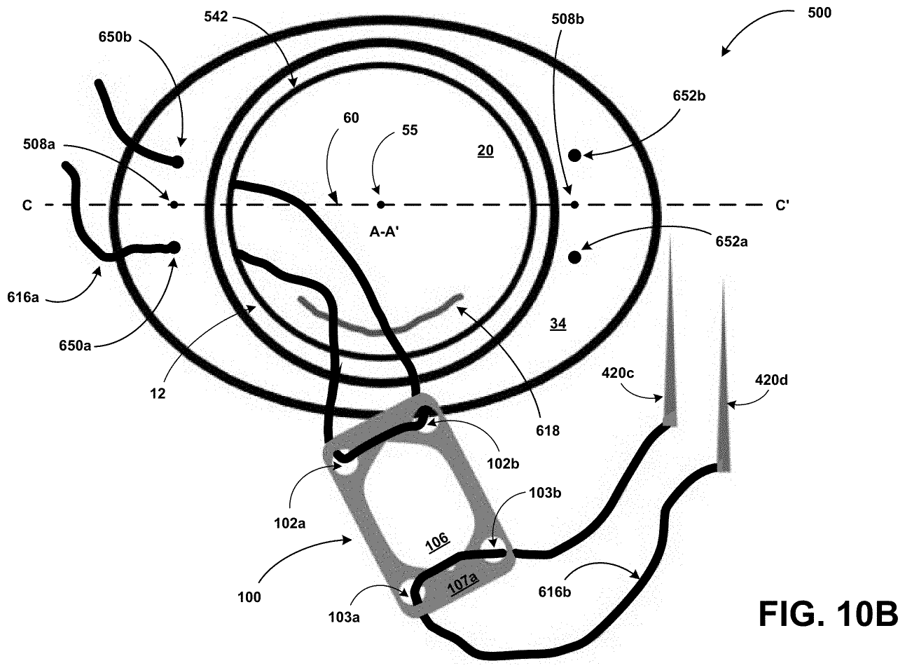

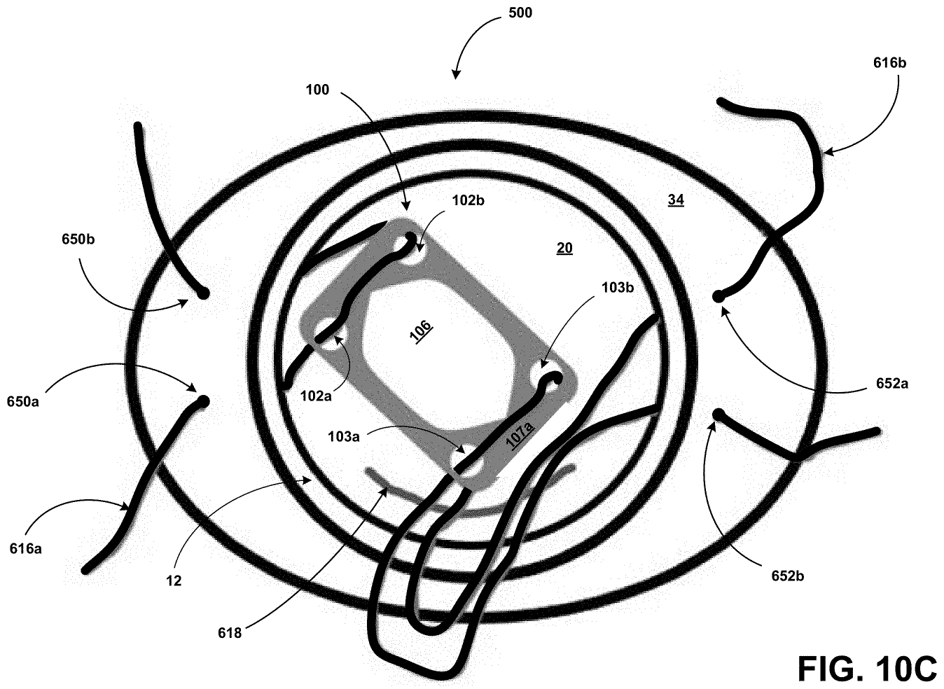

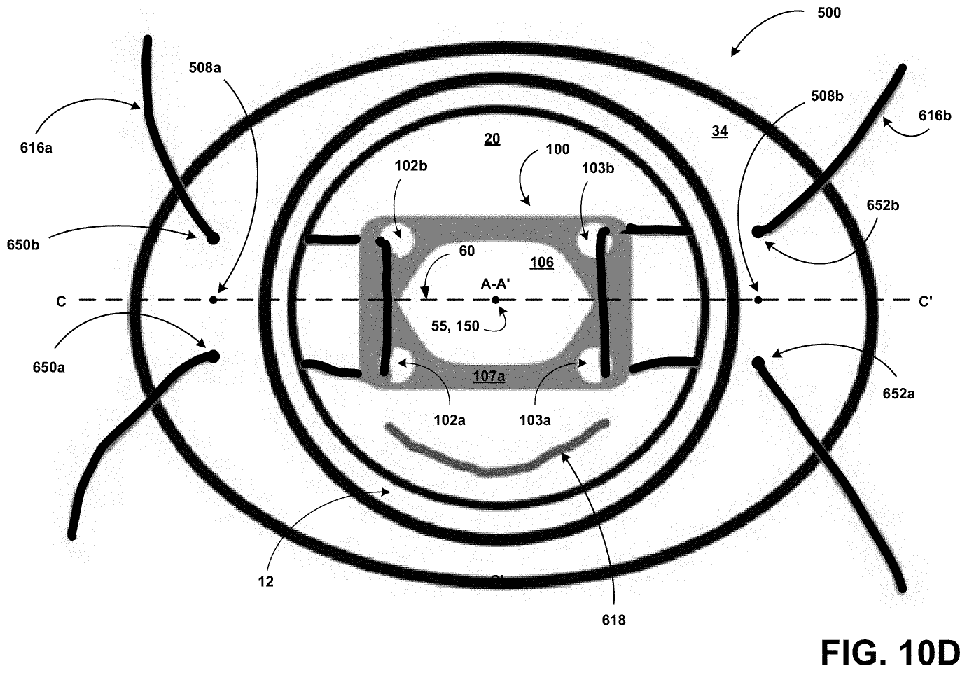

Easy removal also facilitates the use of advanced technology IOLs, like multifocal and trifocal lenses. While these lenses provide a greater range of focus, they are also less forgiving of decentration or retinal issues. Likewise, the ability to rotate the surgical axis 60 in performing the methods of surgical implantation of the invention also permits easier centration of the IOLs with the desired axis of the eye (e.g. the optical axis 55, visual axis 59, or possibly another axis). For example, a multifocal IOL, fixated within the prosthesis of the present invention rather than directly to the iris 12 or sclera 36, can be easily replaced with a mono-focal IOL without causing extensive damage to the supporting structures of the eye 10. Those of skill in the art will appreciate that the methods of surgical implantation of the prosthesis 100, 200 of the invention is not limited to lens replacement necessitated by the surgical removal of cataracts. As is illustrated in FIGS. 10A and B, a desired surgical axis 1510 can be established that matches the axis of astigmatism 900 of an eye 800 to facilitate easier placement of single-piece toric lenses 872 as well.

FIGS. 3A, B illustrate an embodiment 100 of the capsular prosthesis of the invention. In an embodiment, the prosthesis 100 is a thin rectangular sheet 108 of preferably low bio reactive or bioinert, flexible (yet resilient) material having two planar faces 107 that are substantially identical. The sheet has two sets 102a, b and 103a, b of suture apertures through the sheet 108 proximal to its corners or vertices. The sheet 108 has an aperture 106 substantially centered within that is large enough to provide an optical line of sight along optical axis A-A′ 55 for the optics of most commercially available intraocular lenses (IOLs). Center aperture 106 has a centroid 150, that itself can also be the centroid of the sheet 108. Center aperture 106 further includes vertex features 104a, b suitable for capturing haptics of the IOLs passed therethrough to resist them from sliding once captured therein.

In an embodiment, sheet 108 can have a length 110a of approximately 11 mm, a width 110b of approximately 7 mm, and a thickness 110c that can be approximately 0.25 mm. In an embodiment, center aperture 106 can have an internal length of about 8 mm between vertex features 104, and an internal width of about 5 mm. The diameter of suture apertures 102a, b and 103a, b can be about 1.5 mm. Those of skill in the art will recognized that these dimensions may be varied to fit a range of commercially available lenses, sutures, and needles. The thickness 110c of the sheet 108 will vary depending upon the material from which the sheet is made. The sheet can be made of substantially bioinert materials including but not limited to, silicone, polyimide, acrylic or the like. The sheet 108 should be flexible enough that it is foldable, so that it can be made small enough to be inserted into the eye through a primary clear corneal incision of about 2-3 mm. It should also be sufficiently resilient to re-establish its full original dimensions for proper deployment once inserted into the eye. Those of skill in the art will appreciate that the height of sheet 108 will be dictated by the anatomy of the eye. Sheet 108 should be operable to capture and support optic 72, 82 of lens 70, 80, by substantially aligning centroid 76, 86 of optic 72, 82 with centroid 105 of central aperture 106. By substantially aligning center aperture centroid 150 with optical axis A-A′ 55 or visual axis B-B′ 59 during implantation, centroid 76, 86 of IOL should also be substantially so aligned. The sheet 108 does not have to be particularly rigid because it is sutured to be supported at its four vertices, which allows it to be suspended like a trampoline and is therefore maintained at its fully deployed geometry to provide sufficient supportive rigidity within the appropriate plane.

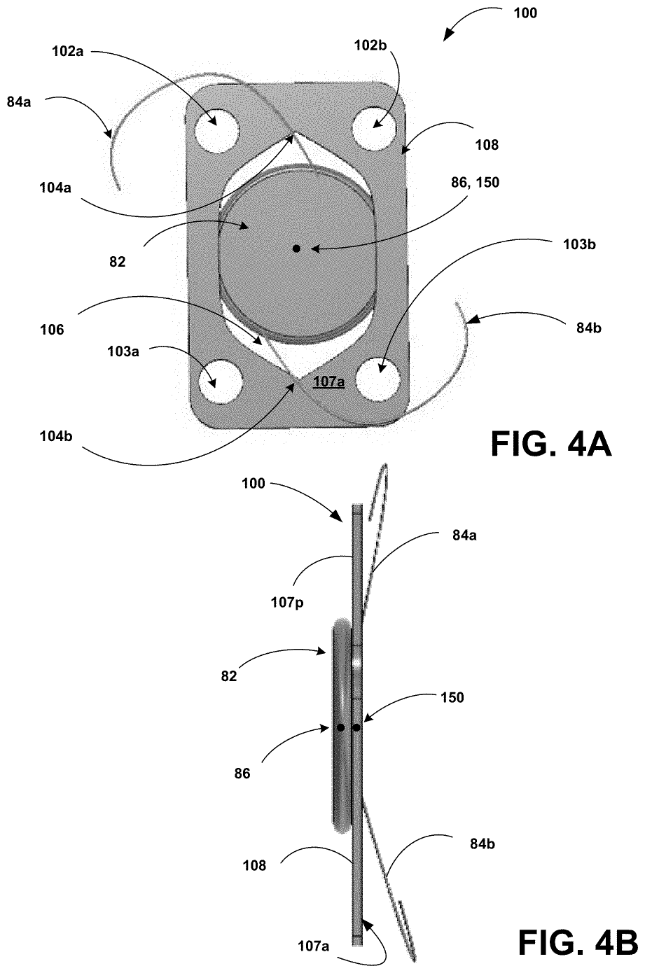

FIGS. 4A, B illustrate a view of what is defined as the anterior surface 107a of the prosthesis 100 from the perspective of a surgeon. The three-piece IOL 80, FIG. 2B mounted on the prosthesis 100 having optic 82 and haptics 84a, b. The IOL 80 is mounted in a reverse optic capture configuration, with its haptics 84a, b placed forwardly through aperture 106 from the posterior side and captured within vertex features 104a, b respectively on the anterior surface 107a. Optic 82 is substantially centered behind center aperture 106 on what is the posterior surface 107p from the perspective of a surgeon and has optical line of sight along axis A-A′ 55. FIG. 4A shows a surgical axis C-C′ 60 aligned with the optical axis A-A′ 55.

FIG. 4B shows a side view of the reverse captured IOL 80, whereby the optic 82 has been prolapsed through the aperture 106 such that haptics 84a, b exert a force on the anterior surface 107a that pulls the optic 82 against the posterior surface 107p of the sheet 108. This works much the same way as does a sulcus placement of such a lens using reverse optic capture, wherein the optic 82 is prolapsed into the capsulorhexis 40 into the anterior capsule 34, the haptics 84a, b disposed in the sulcus 18 and pulling the optic 82 against the inside surface of the anterior capsule 34 defining the capsulorhexis 40.

FIG. 5 illustrates an alternate embodiment 200 of the prosthesis of the invention that is triangular in geometry rather than rectangular. This triangular embodiment 200 has three, rather than four, suture apertures 202, 203a and 203b each located proximally to one of the three vertices of the triangular sheet 208. The three vertices 202, 203a and 203b are rounded off to avoid creating sharp points that could irritate or potentially damage structures in the eye during implantation. The thin rectangular sheet 208 is also made of a bioinert, flexible (yet resilient) material having two planar faces 207 that are substantially identical. The triangular sheet 208 has an aperture 206 substantially centered within that is large enough to provide an optical line of sight along a desired axis of the eye 50, (e.g. optical axis A-A′ 55 or visual axis B-B′ 59 for the optics of most commercially available intraocular lenses (IOLs). Center aperture 206 has a centroid 250 that itself can also be the centroid of the sheet 208, and further includes vertex features 204a, b suitable for capturing haptics of the IOLs passed therethrough to resist them from sliding once captured therein. In an embodiment, the dimensions of the triangular sheet 208 can be scaled as necessary to accommodate the inside dimensions of the center aperture 206.

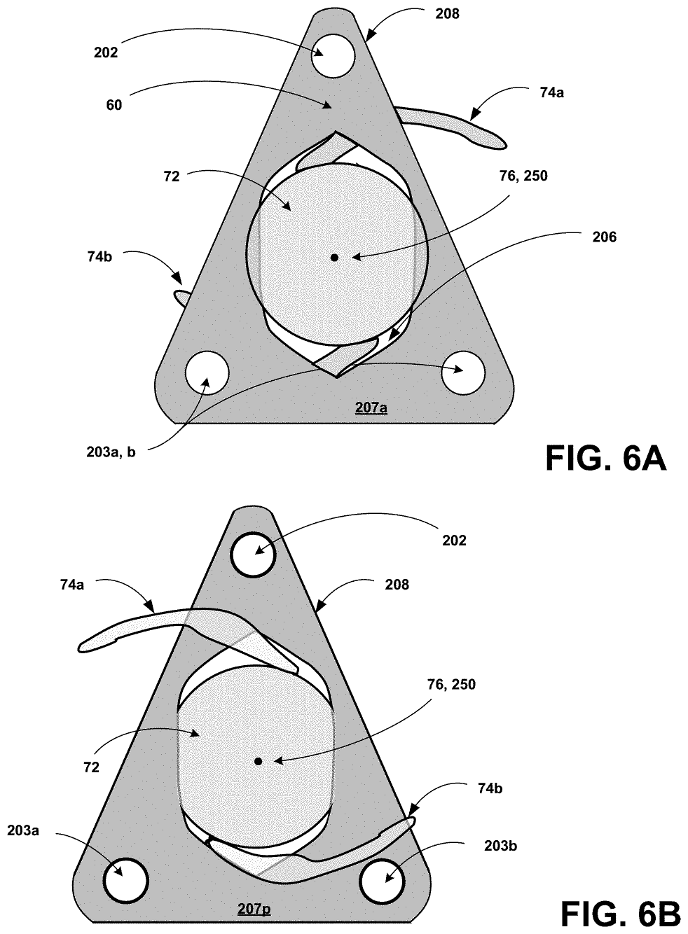

FIG. 6A illustrates a view of what is defined as the anterior surface 207a of the prosthesis 200 from the perspective of a surgeon. A one-piece IOL 70, FIG. 2A is mounted on the prosthesis 200 having optic 72 and haptics 74a, b. The IOL 70 is shown mounted on prosthesis 200 using an optic capture, where its haptics 72a, b are placed rearwardly through aperture 206 and emerging from the aperture 206 and captured within vertex features 204a, b respectively on the posterior side and surface 207p. The centroid 76 of optic 72 is substantially centered with the centroid 250 of aperture 206 on what is the anterior surface 107a from what will be the perspective of a surgeon performing implantation of the centroid 76 of optic 72 is substantially centered with optical axis A-A′ 55 of an eye 50. Those of skill in the art will appreciate that centration of the optic 72 and centroid 250 can be made with respect to any desired axis, including the visual axis B-B′ 59. FIG. 6B shows a posterior view of the captured IOL 70, whereby the haptics 74a, b have been prolapsed through the aperture 206 from the anterior side such that haptics 74a, b exert a force on the posterior side surface 207p that pulls the optic 72 against the anterior surface 107a of the sheet 208 and maintains its position.

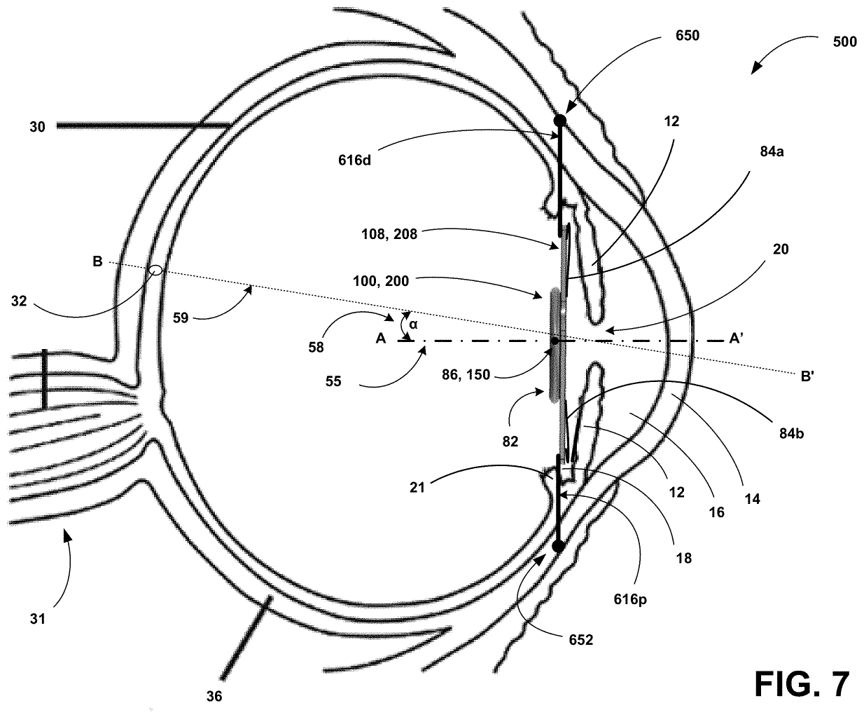

FIG. 7 illustrates prosthesis 100 and a reverse optically captured three-piece IOL having optic 82 and haptics 84a, b mounted thereon, having been implanted in the posterior chamber 18 of eye 500 within the space that was once the approximate location of the anterior capsule 34 in accordance with an embodiment of surgical methods of the invention. Two looped transscleral sutures 616p (i.e. posterior) and 616d (i.e. distal) are shown each a pair of ends, which are passed through sclera 36 at sclerotomies 652a, b and 650a, b respectively, and looped through the suture apertures 103a, b and 102a, b of prosthesis 100 respectively. Each pair of ends of the looped sutures 616p, d respectively is ultimately surgically secured to the sclera 36 at paired sclerotomy points 652a, b and 650a, b (at the bottom and top the eye 500 respectively). Sutures 616d, p should be of a thickness, strength and durability sufficient to hold an IOL in place permanently (e.g. a 9-0 or larger prolene, or Gore-Tex suture).

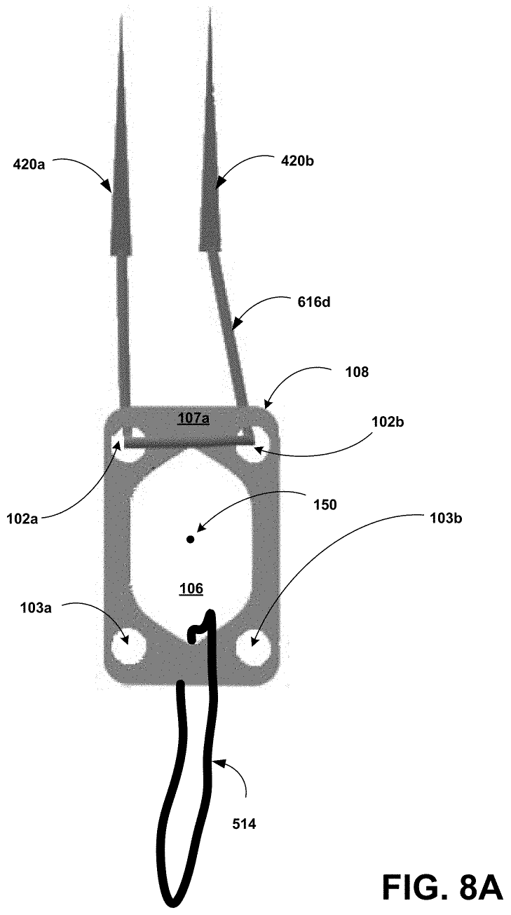

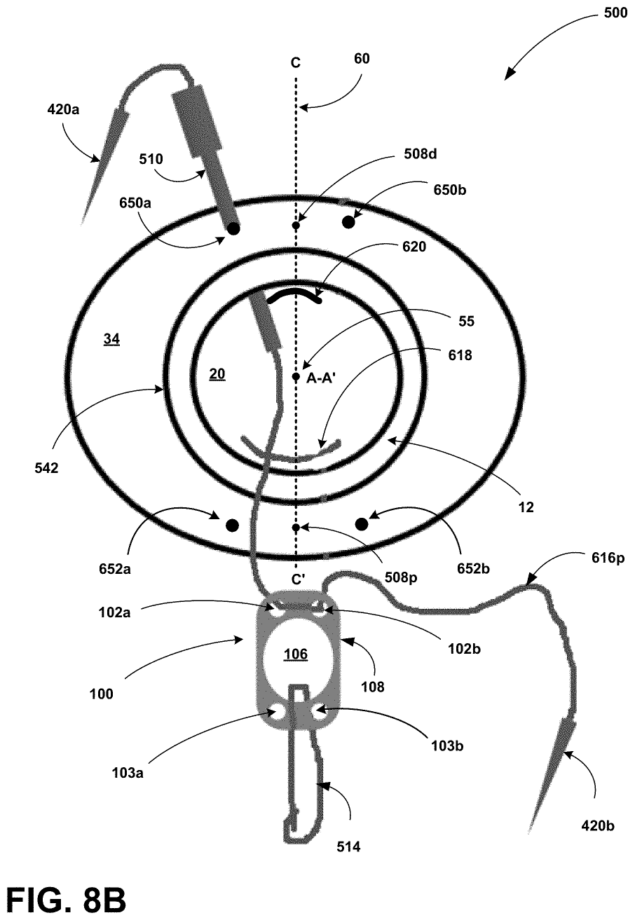

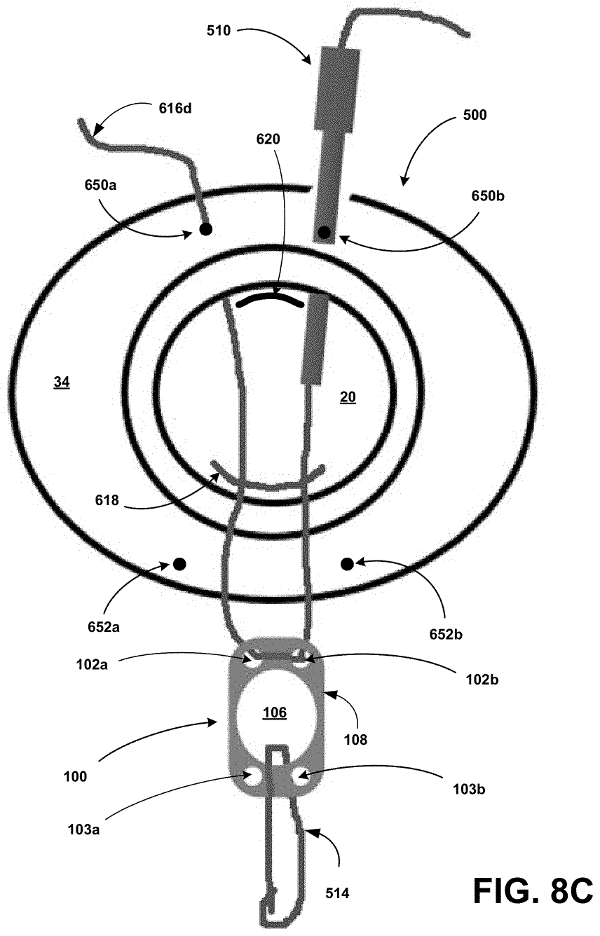

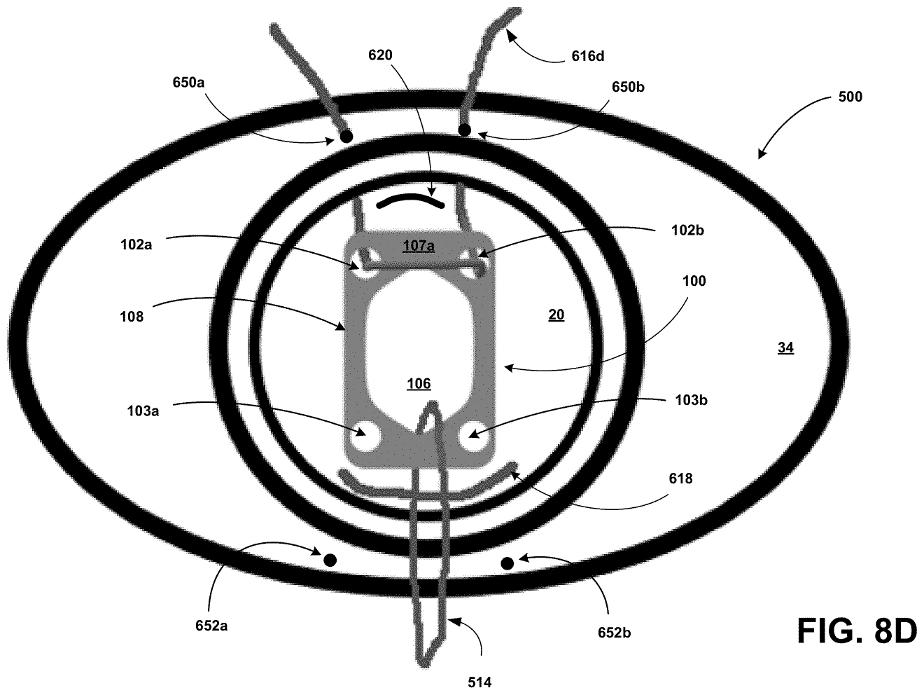

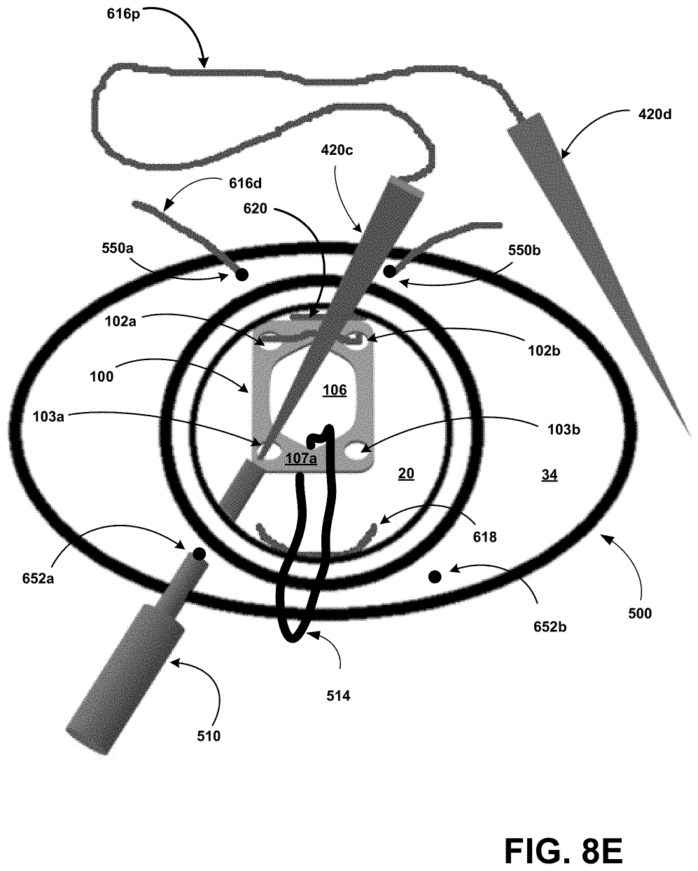

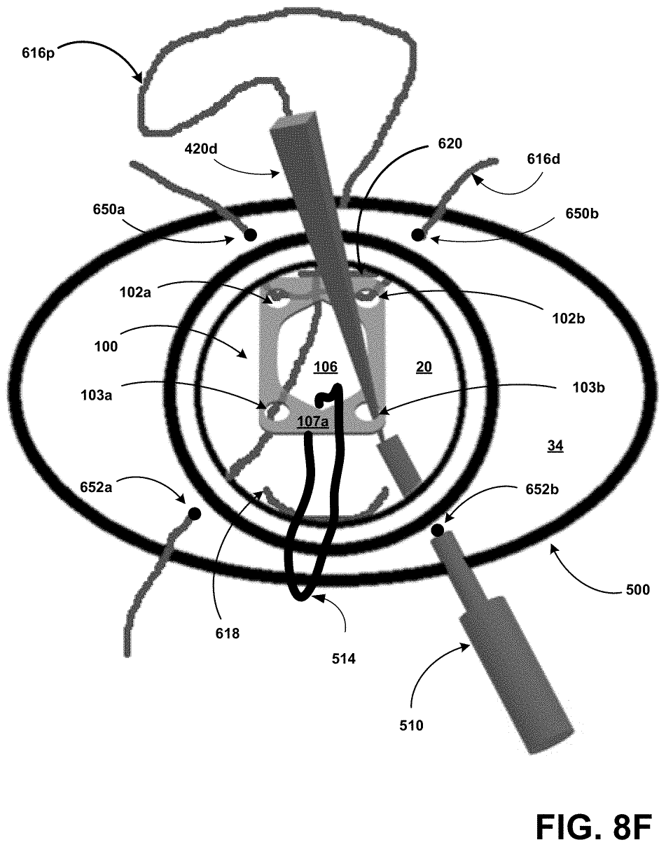

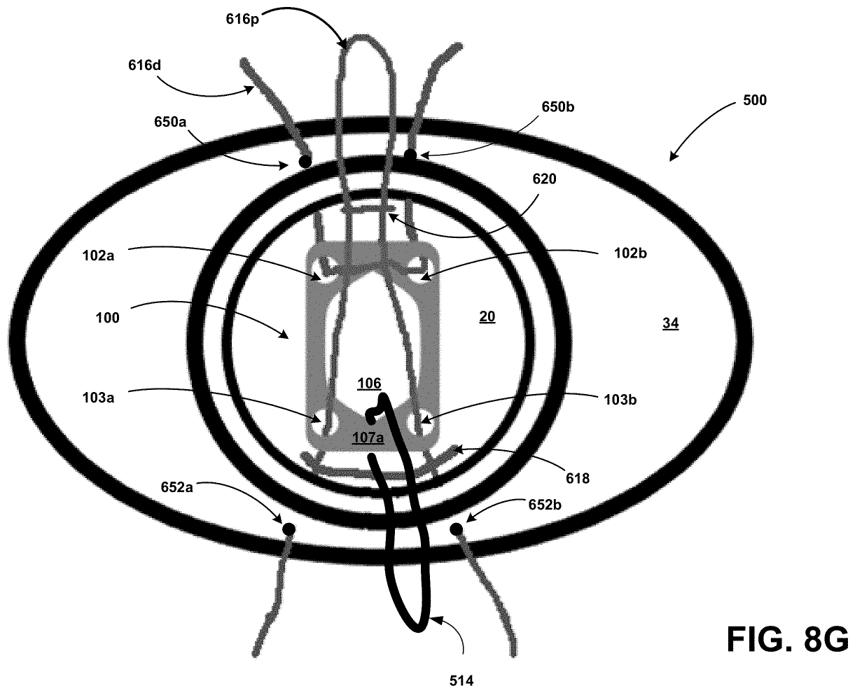

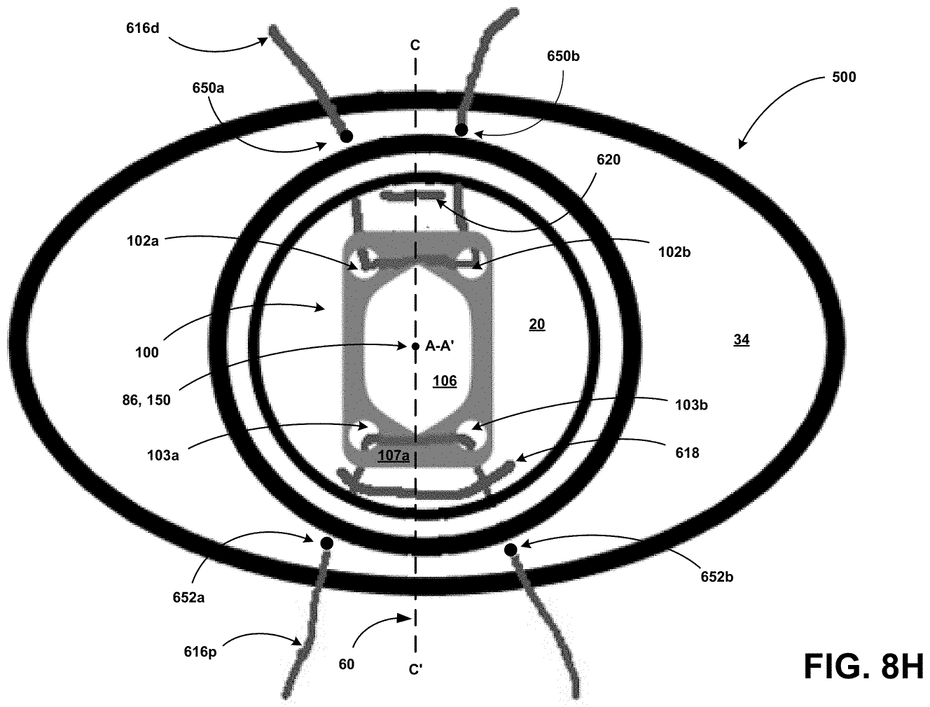

FIGS. 8A-J illustrate surgical steps of one embodiment of a method of surgical implantation of the prosthetic 100, 200. These steps are now described with reference to those illustrations. Prior to beginning surgery, the surgeon will assure that the eye 500 is of appropriate pressure using either viscoelastic solution or infusion.

In an embodiment, distal suture 616d is initially established as a double armed suture (needles coupled to both ends of a loop of suture) with long needles 420a, 420b (e.g. CTC-type needles) as illustrated in FIG. 8A. Distal suture 616d is coupled to one end (which becomes the distal end) of the prosthesis 100, by pulling one needle 420a through one suture aperture 102a of prosthesis 100 and the second needle 420b through the second suture aperture hole 102b so that a loop of suture is slidably secured to the distal end of the sheet 108 of prosthesis 100. If embodiment 200 of prosthesis 100 is used, this distal suture 616d can be looped through single aperture 200 or through apertures 203a, b, depending upon the desired orientation of the prosthesis 200. A loose loop of suture 514 is also established through the lens aperture 106, 206 at the proximal end of the prosthesis 100, 200. Loop 514 is of sufficient length to serve as a safety suture to prevent the prosthesis 100, 200 of the invention from falling into the vitreous of the eye 500 like a trap door during the surgery.

As illustrated in FIG. 8B, a primary clear corneal incision 618 of about 3 mm is made along the predetermined surgical axis C-C′ 60 falling in the plane in which the IOL 70, 80 is to be placed. A secondary clear corneal incision 620 of about 1 mm is made substantially 180 degrees from the primary incision and along predetermined surgical axis C-C′ 60. The plane in which the predetermined surgical axis C-C′ 60 lies also intersects both optical axis A-A′ 55 and visual axis B-B′ 59. Those of skill in the art will appreciate that the primary 618 and/or secondary 620 clear corneal incisions could be made in the sclera 36 instead of the cornea 14 if preferable.

A distal mark 508d is first determined and then made on the surface of sclera 36 by measuring along the surgical axis C-C′ 60 extending above the center of the pupil 20 to a point on sclera 36 about 4 mm posterior to the surgical limbus 542 of the eye 500, and which is also just posterior (with respect to the pupil 20) to the secondary clear corneal incision 620. Distal sclerotomy points 550a, 550b are marked on the sclera 36 to form two ends of a line segment of about 6 mm in length, running through second measured mark 508d and running substantially perpendicular to the predetermined surgical axis C-C′ 60 such that predetermined surgical axis C-C′ 60 bisects the line segment that connects the two sclerotomy points 550a, 5520. The surgical limbus 542 of the eye 500 forms the border between the transparent cornea and opaque sclera 36, contains the pathways of aqueous humor outflow, and is the site of surgical incisions for cataract and glaucoma (hence being referred to as the surgical limbus).

A proximal mark 508p is then first determined and then made on the surface of the sclera 36 by measuring along the surgical axis C-C′ 60 extending below the center of the pupil 20 a point on sclera 36 about 4 mm posterior to the surgical limbus 542 of the eye 500, and which is just posterior (with respect to the pupil 20) to the primary clear corneal incision 618. Proximal sclerotomy points 552a, 552b are marked on the sclera 36 to form two ends of a line segment of about 6 mm in length, running through second measured mark 508p and running substantially perpendicular to the predetermined surgical axis C-C′ 60 such that predetermined surgical axis C-C′ 60 bisects the line segment that connects the two sclerotomy points 552a, 552b.

As illustrated in FIG. 8B, a 27 gauge or similar hollow hypodermic or sclerotomy needle 510 can be used to make a sclerotomy at a first 550a of the two marks until the needle 510 becomes visible behind the pupil 20 of the eye 500. Using one end of the preloaded double armed suture 616d, the CTC needle 420a is inserted through the primary incision 618 into the eye 500 and docked into the sclerotomy needle 510. Loading of the CTC needle 420a continues until its tip is well outside the eye 500 as shown. The needle 510 is then removed the CTC needle 420a is pulled until that first paired end of the suture 616d is entirely through the sclera 36. Needle 420a is also removed from the first paired end of suture 616d as is shown in FIG. 8C.

The foregoing steps are then repeated for the second sclerotomy mark 550b as illustrated in FIG. 8C. The hollow hypodermic needle 510 can be used to make a sclerotomy at the second 550b of the two sclerotomy marks until the needle 510 becomes visible behind the pupil 20 of the eye 500. Using the second paired end of the preloaded double armed suture 616d, the second CTC needle 420b is inserted through the primary incision 618 and into the eye 500 and is docked it into the sclerotomy needle 510. The CTC needle 420b is then loaded until its tip is well outside the eye 500 as described above. Hollow needle 510 is then removed and the CTC needle 420b is pulled through until the second paired end of the suture 616d is entirely through the sclera 36. Needle 420b is also removed from the first end of suture 616d as shown in FIG. 8D.

As is also illustrated in FIG. 8D, both ends of the looped transscleral suture 616d have been pulled, thereby having caused the prosthesis 100 of the invention to be drawn into the eye 500 through the primary incision 618. Those of skill in the art will appreciate that the drawings herein are not to scale, and that the width 110b of the sheet 108 could be over twice the length of the 2-3 mm primary incision 618. As previously discussed, the sheet 108 will be sufficiently flexible such that it easily could be folded in half and held in that mode to facilitate insertion through the primary incision 618 before releasing it to re-establish its full form. Those of skill in the art will further appreciate that the loop suture 514 provides a means by which to maintain sheet 108 of the prosthesis 100 in a substantially planar orientation with respect to the surgical axis C-C′ 60. Loop 514 remains at least partially outside of the eye 500 through the primary incision 618 and serves to prevent the prosthesis 100 from falling into the vitreous. It also serves to provide a handle by which to hold the sheet planar while cannulating surgical needles 420c, d through the proximal suture apertures 103a, b as described below.

As illustrated in FIG. 8E, a second double armed suture 616p has been established (preferably but not necessarily when the first double armed suture 616d was established) having CTC needles 420c and 420d attached at the first and second ends thereof. The hollow hypodermic needle 510 can then be used to make a sclerotomy at mark 552a until the needle 510 becomes visible behind the pupil 20 of the eye 500. Using the first end of the second preloaded double armed suture 616p, the CTC needle 420c is inserted through the secondary incision 620 and into the eye 500 and is first cannulated through suture aperture 103a and then docked into the sclerotomy needle 510 as shown. The CTC needle 420c is loaded until its tip is well outside the eye 500 as previously discussed. The needle 510 is removed, and the CTC needle 420c is pulled until that first paired end of the suture 616p is entirely through the sclera 36. Needle 420c is also removed from the first end of suture 616d as is shown in FIG. 8F.

And as is further illustrated in FIG. 8E, the hollow hypodermic needle 510 can be used to make a sclerotomy at mark 552b until the needle 510 becomes visible behind the pupil 20 of the eye 500. The second paired end of the second preloaded double armed suture 616p coupled to CTC needle 420d is inserted through the secondary incision 620 and into the eye 500 and is first cannulated through suture aperture 103b and then docked it into the sclerotomy needle 510 as illustrated. The CTC needle 420d is loaded until its tip is well outside the eye 500 as previously discussed above. The hollow needle 510 is removed, and the CTC needle 420d is pulled until that end of the suture 616p is entirely through the sclera 36. Needle 420c has also removed from the first end of suture 616d. The result is shown in FIG. 8G.

The loop suture 514 can now be removed from the center aperture 106 of the prosthesis 100 and both of the paired ends of the proximal transscleral suture 616p are pulled to suspend the prosthesis 100 within the eye 500 as is illustrated in FIG. 8H. Both ends of the distal 616d and proximal 616p looped sutures can be pulled simultaneously to adjust the position of the prosthesis 100 along the surgical axis 55 C-C′ 60 as needed to substantially center the centroid 150 of center aperture 106 of the prosthesis 100 to the optical axis A-A′ 55 (approximately the center of the pupil 20) of the eye 500. This result is illustrated by FIG. 8H.

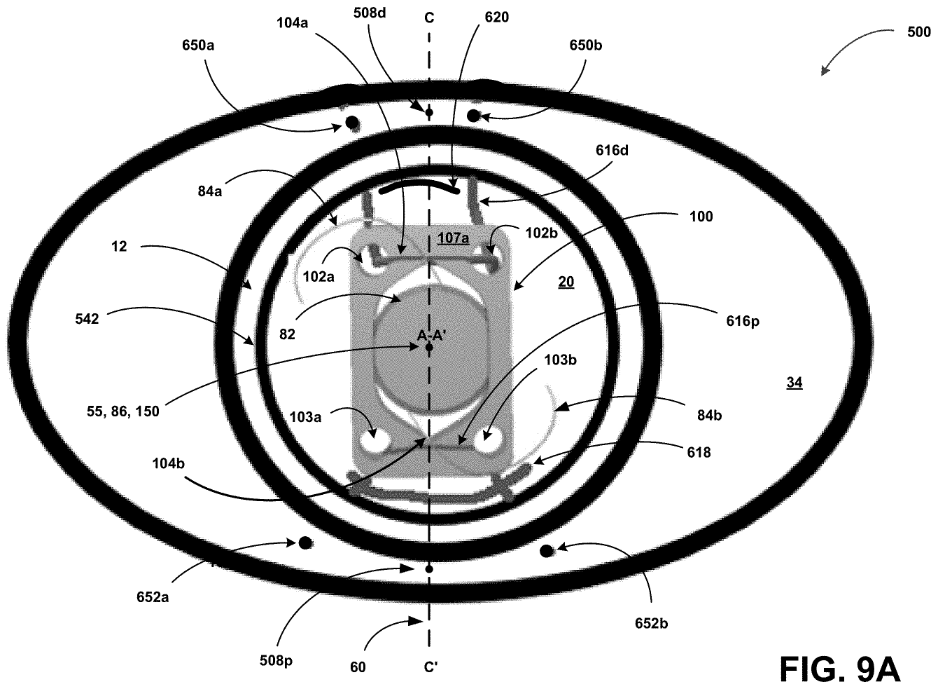

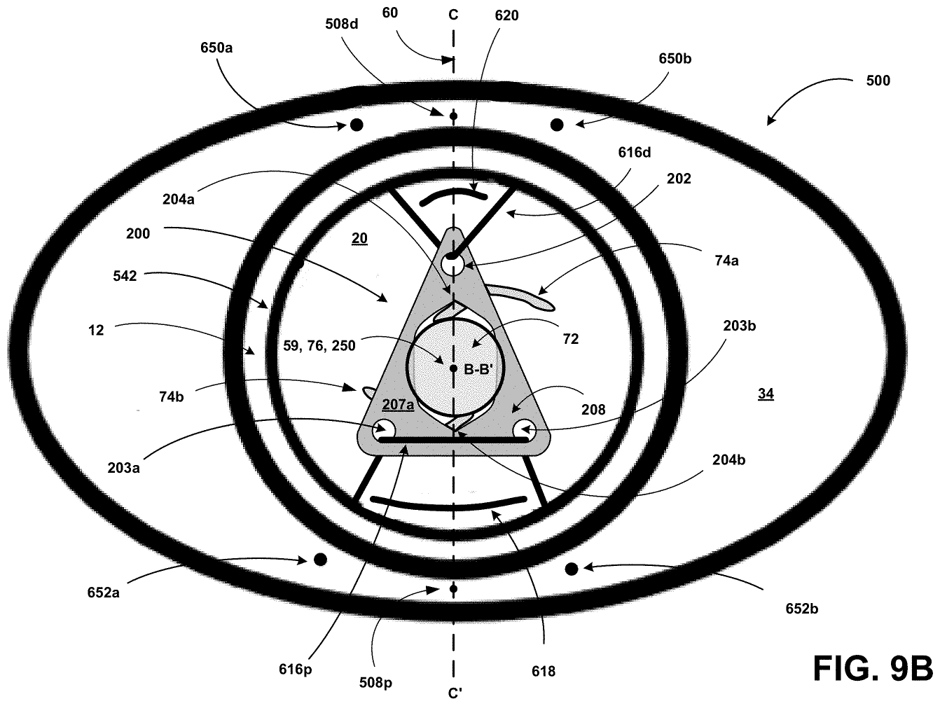

As illustrated in FIG. 9A, with the prosthesis 100 centered so that the , the paired ends of each of the transscleral looped sutures 616d, 616p can then either be tied, or subjected to heat cautery to make thickened flanges, to secure the sutures within the sclera 36. With the prosthesis 100 now securely centered within the eye 500, a one or three piece intraocular lens 70, 80 can be inserted into the eye through primary incision 618 using a standard lens insertion cartridge (not shown) known to those of skill in the art. FIG. 9 illustrates a three-piece IOL 80 that has been mounted to prosthesis 100 by way of reverse optic capture. A Sinskey hook or other instrument can be used as known in the art to manipulate the optic 82 so that its longitudinal edges are posterior to the prosthesis 100 and in contact with a posterior facing surface (107b not shown) of the sheet 108 of the prosthesis 100, leaving the haptics 84a, b anterior to the prosthesis 100 and captured within the vertex features 104 on the anterior face 107a. The position of optic 82 is then adjusted until the optic centroid 85, the central aperture centroid 150 and the optical axis A-A′ 55 are substantially aligned.

In FIG. 9B, surgical implantation of embodiment 200 of the prosthesis is illustrated. The only difference in the surgical process presented above is that because embodiment 200 is triangular, one of the transscleral sutures is looped through the single suture aperture 202. In addition, alignment is made to the visual axis B-B′ 59. The single aperture 202 can be at the distal end of the prosthesis, or it can be rotated 180 degrees so that it is located at the proximal end. FIG. 9B illustrates a single-piece IOL 70 that has been mounted to prosthesis 100 by way of optic capture. Thus, a Sinskey hook or other instrument can be used as known in the art to manipulate the optic 72 so that its longitudinal edges are anterior to the prosthesis 200 and in contact with an anterior facing surface 107a of the sheet 208 of the prosthesis 200, leaving the haptics 74a, b posterior to the prosthesis 100 and captured within the vertex features 104 on the posterior face 107b (not shown). The position of optic 72 is then adjusted until the optic centroid 76, the central aperture centroid 250 and the visual axis B-B′ 59 are substantially aligned. Those of skill in the art will appreciate that each embodiment of the prosthesis 100, 200 is capable of supporting either type of IOL 70, 80.

As illustrated in FIG. 7, the plane to be occupied by the prothesis 100, 200 contains the desired surgical axis C-C′ 60 and is shown to be substantially perpendicular to the optical axis A-A′ 55. Transscleral Sutures 616d, 616p defined by the proximate 552a, b and distal 550a, b pairs of sclerotomy points are located anterior to the ciliary bodies 21. This of course defines the relative position of the optic 72, 82 along the optical axis A-A′ 55. Those of skill in the art will recognize that other locations for the sclerotomy points can range to just posterior to the ciliary bodies 21 at the anterior pars plana), resulting in different positions of the optic 70, 80 along the optical axis A-A′ 55 may be also desirable.

Moreover, those of skill in the in art will appreciate the desired surgical axis C-C′ 60 forms a substantially perpendicular bisector of each pair of sclerotomies 652a, b and 650a, b, and are therefore approximately 180 degrees apart from one another along the desired surgical axis C-C′ 60. These points can be rotated over 180 degrees before returning to the functionally equivalent original (albeit inverted) orientation as shown in FIG. 7. The resulting rotation around the optical axis A-A′ 55 does not affect centration of spherical lenses, but this can be useful with regard to the fixation of non-spherical lenses such as toric lenses, the rotational orientation of which is critical for correcting a person's astigmatism. This will be discussed in more detail below.

Those of skill in the art will also appreciate that the establishment of sclerotomy fixation points for the prosthesis can also be rotated forward to center the IOL's on the visual axis if desirable. The embodiment of the surgical method described above establishes the surgical axis C-C′ 60 to be substantially perpendicular to the optical axis A-A′ 55. This is the easier axis to which surgeons can achieve centration because it substantially aligns with the center of the pupil 20. But in the event that centration of the IOL 70, 80 with visual axis visual axis B-B′ 59 is desirable, the surgical methods and the prosthesis 100, 200 of the invention can easily accommodate rotating the plane in which the prosthesis 100, 200 lies to be made more perpendicular to the visual axis B-B′ 59 by rotating the surgical axis C-C′ 60 forward by an angle substantially equal to the angle α 58 shown in FIG. 7.

An alternative embodiment of the surgical method discussed above can eliminate the need to cannulate the second transscleral suture 616p within the eye 500, and further eliminates the need for secondary incision 620. In this embodiment, both transscleral double armed sutures 616a, b can be looped through the suture apertures of prosthesis 100, 200 outside of the eye, as illustrated in FIG. 10A. The predetermined surgical axis C-C′ 60 has been rotated counterclockwise 90 degrees and is still perpendicular to the optical axis A-A′ 55. In this surgical method, the primary incision 618 is bisected by an axis of incision 61 that is substantially perpendicular (i.e. at 90°) to the predetermined surgical axis C-C′ 60.

A first surgical mark 508a is determined and then made on the surface of sclera 36 by measuring along the surgical axis C-C′ 60 extending left of the center of the pupil 20 to a point on sclera 36 about 4 mm posterior to the surgical limbus 542 of the eye 500, and which is also just posterior (with respect to the pupil 20) to a radius including the secondary clear corneal incision 620. A first pair of sclerotomy points 550a, 550b are marked on the sclera 36 to form two ends of a line segment of about 6 mm in length, running through second measured mark 508b and running substantially perpendicular to the predetermined surgical axis C-C′ 60 such that predetermined surgical axis C-C′ 60 bisects the line segment that connects the two sclerotomy points 550a, 550b. Those of skill in the art will appreciate that in this embodiment of the surgical procedure of the invention, it is not important which of the transscleral sutures 616a, b is established first, nor for that matter whether the surgical axis C-C′ 60 has been considered to have been rotated clockwise or counterclockwise.

A second surgical mark 508b is then determined and made on the surface of the sclera 36 by measuring along the surgical axis C-C′ 60 extending to the right of center of the pupil 20 to a point on sclera 36 about 4 mm posterior to the surgical limbus 542 of the eye 500, and which is just posterior (with respect to the pupil 20) to a radius including the primary clear corneal incision 618. Proximal sclerotomy points 552a, 552b are marked on the sclera 36 to form two ends of a line segment of about 6 mm in length, running through second measured mark 508b and running substantially perpendicular to the predetermined surgical axis C-C′ 60 such that predetermined surgical axis C-C′ 60 bisects the line segment that connects the two sclerotomy points 552a, 552b.

A 27 gauge or similar hollow hypodermic or sclerotomy needle 510 can be used to make a sclerotomy at a first 550a of the two marks 550a, 550b until the needle 510 becomes visible behind the pupil 20 of the eye 500. Using one end of the first preloaded double armed transscleral suture 616a, the CTC needle 420a is inserted through the primary incision 618 into the eye 500 and docked into the sclerotomy needle 510. Loading of the CTC needle 420a continues until its tip is well outside the eye 500 as shown. The hollow needle 510 is then removed and the CTC needle 420a is pulled until that first paired end of the suture 616d is entirely through the sclera 36. Needle 420a is also removed from the first paired end of suture 616d as is shown in FIG. 10A. The steps are then repeated for the second of the paired ends of transscleral suture 616a, as is also illustrated in FIG. 10A.

The foregoing steps are then repeated for the second transscleral suture 616b as illustrated in FIG. 10B, where sclerotomies are made at points 652a, b and needles 420c, d are cannulated through the hollow needle 520. Needles 420a, b, c, d are decoupled from each of the paired ends. The paired ends of the first transscleral suture 616a can be pulled to bring the prosthesis 100, 200 into the eye 500 through incision 618 before the second transscleral suture 616b is fixed, in which case portions of the second transscleral suture 616b can remain protruding from the incision 618 to keep the prosthesis suspended as illustrated in FIG. 10C, just as the loop 514 did in the first embodiment of the surgical method described above. The paired ends of the second transscleral suture 616b are then pulled to suspend the prosthesis in the eye 500. In the alternative, the prosthetic can remain outside the eye 500 until both sutures 616a, b are placed. The sheet 108, 208 of prosthesis 100, 200 can be folded in half with a forceps and inserted through the incision 618, and the paired ends of both of the transscleral sutures 616a, b can be pulled to suspend the prosthesis in the eye 500.

As is the case with the first embodiment of the surgical method, the two paired ends of each suture 616a, b can then be pulled to adjust and substantially center the center aperture 106 of prosthesis 100, 200 to the optical axis A-A′ 55 (or the visual axis visual axis visual axis B-B′ 59 if desirable), depending upon the angle of the predetermined surgical axis surgical axis C-C′ 60, as illustrated in FIG. 10D. The sutures can then be fixed as previously discussed in the sclera 36.

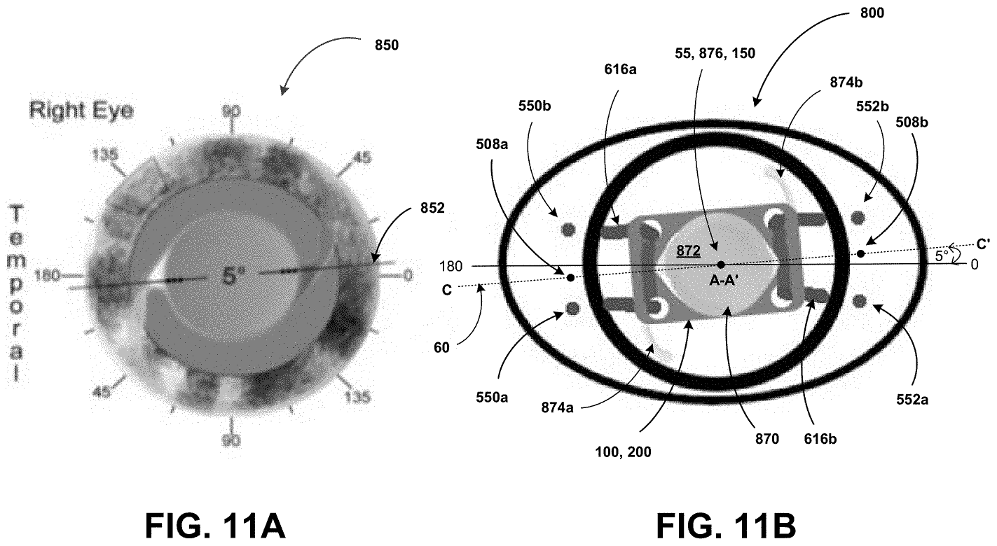

Thus, embodiments of the surgical method of the invention permit the prosthesis 100, 200 of the invention to be surgically implanted at any predetermined angle of orientation of the surgical axis C-C′ 60 over the 360° around virtually any axis, but particularly the optical axis A-A′ 55 or the visual axis B-B′ 59. This makes implantation of non-spherical lenses, such as a toric lens 870 that is designed to correct a person's astigmatism easier to implement. FIG. 11A presents a visual representation of a patient's astigmatism commonly produced by a diagnostic instrument. The astigmatism is presented as an angled axis of astigmatism 852 centered on the optical axis of the eye 800. FIG. 11B represents implantation of the prosthesis 100, 200 using a surgical axis C-C′ 60, predetermined to be substantially the same as the axis of astigmatism 852.

By orienting the prosthesis 100, 200 in accordance with the axis of astigmatism 852, the surgeon does not have to provide a correct orientation of the non-spherical lens. The surgeon must only orient the tonic lens optic 872 with the center aperture 106 of the prosthesis, in accordance with standard orientation established by the manufacturer for optic capture within the prosthesis 100, 200. The standard orientation of the IOL 870 can be normalized to that disclosed in FIGS. 6A, 6B and 9B, with the haptics 874a, b aligned to be captured by vertex features 104, 204 of prosthesis 100, 200. Because toric lenses 870 are typically single piece lenses, they will be captured using optic capture as is also illustrated in FIGS. 6A, 6B and 9B and 11B.

Those of skill in the art will recognize that certain modifications of the embodiments disclosed herein can be made without exceeding the intended scope of the invention. Modifications to the geometry of the prosthesis 100, 200, the physical dimensions and the number of suture apertures can also be varied and will still be within the intended scope of the invention, as long as such geometries and dimensions provide sufficient points of contact that can produce the requisite stability of the prosthesis once implanted, as well as providing the requisite substantially centered alignment of the optical 55 or visual 59 axis of the eye with IOL optics 72, 82 captured thereon. For example, the geometry of the prosthesis could be hexagonal, pentagonal, or even star shaped. Additional vertices could also be provided along the sides of rectangular prosthesis 100 without changing its geometry. The increased numbers of vertices of the geometry could provide additional suture apertures if desirable, which would lead to additional points of contact and greater stability. While the number of transscleral sutures 616 should be kept to a minimum to simplify the procedure, additional points of contact may be desirable.

The minimum points of contact necessary to prevent rotation of the prosthesis 100, 200 can be provided through at least two transscleral sutures 616 providing at least three points of contact between the sclera 36 of the eye 500 and prosthesis 100, 200 through apertures 102, 103 or 202, 203. Any lesser number could lead to undesired rotation of the implanted prosthesis, and therefore the IOL 70, 80, 870. When implanted as illustrated in FIG. 9B, the three apertures 202, 203a, b of triangular embodiment 200 of the prosthesis 100, 200 (located proximally to its vertices) is an example of a geometry providing a minimum number of three points of contact. While there are actually four attachment points provided by the paired ends of the first and second looped transscleral sutures 616d, p, the points of contact as referred to herein are made with reference to the prosthesis 100, 200 itself. The second suture 616p provides two of the points of contact with the prosthesis 200, because it is looped through the two paired apertures 203a, b.

The rectangular embodiment 100 of prosthesis 100, 200, when implanted as illustrated in FIG. 9A, has two paired suture apertures (102a, b and 103a, b) located proximally to each one of its four vertices, and thus increases the number of points of contact between it and the sclera 36 through the sutures 616d, p to four. It will be appreciated that by looping both transscleral sutures 616d, p through paired apertures 102a, b and 103a, b respectively, it will be appreciated that stability has been increased without adding additional sutures 616, and therefore the number of sclerotomies required for implantation.

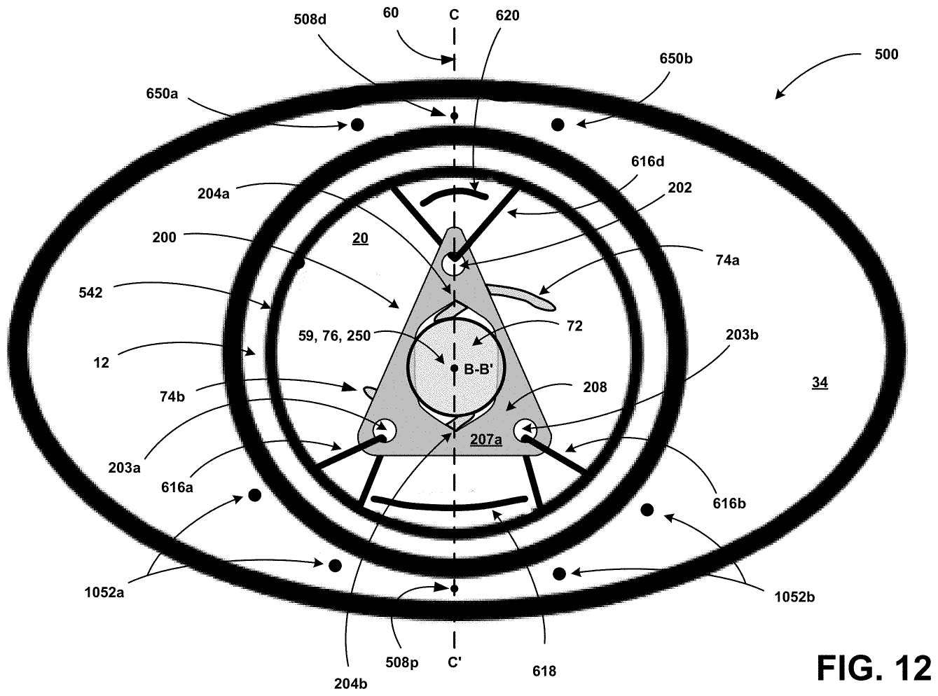

It will be further appreciated in view of FIG. 12 that, while limiting the number of transscleral sutures 616 is preferable with regard to simplifying the procedure and limiting its invasiveness, greater stability for the implanted prosthesis 100, 200 may be achieved if each aperture has its own transscleral suture 616 looped therethrough, rather than looping through pairs of apertures as described above. In this case, rather than marking just two scleral points on either side of the surgical axis 60 as described for embodiments of the surgical method above, two pairs of such points 1052a, b could be made, so that each aperture has its own suture 616 as illustrated.

It will be further appreciated that rather than looping each suture 616 of FIG. 12, the same number of points of contact could be provided to the sclera using single sutures instead. If the diameters of the apertures 202, 203a, b are reduced, each suture 616 could be cannulated through the smaller diameter aperture and constrained therein by creating a flange at the end to create a flange that prevents the suture from slipping back through. Doing so would accomplish reducing the number of sclerotomies required from four to three. These sutures could be affixed to the apertures pre-surgery and would still permit centration adjustment before fixation to the sclera 36. It will be appreciated that the sclerotomy points would be in substantially the same place as the markers established along the surgical axis. It will be further appreciated that this could also be accomplished with one looped suture passed through the base vertices 203a, b of triangular embodiment 200 and a flanged suture pre-secured to vertex 202. Finally, it will be appreciated that the flanged longitudinal sutures will not decrease the number of sclerotomies for those geometries of prosthesis 100, 200 having even numbers of vertices that can be at least paired with a looped suture.

It should be noted the precise implanted position in the space posterior to the sulcus 18 along the optical visual axis A-A′ 59 55 or visual 59 visual axis B-B′ 59 axis can also vary, provided the IOL 70, 80 is compensated as necessary to provide the proper focal length for satisfactory resolution of the image on the retina 30. Additionally, when performing embodiments of the surgical method of the invention, it will be appreciated that certain steps can be performed in a different order from that disclosed without impacting the ultimate result achieved. For example, the marks 508 made along the surgical axis 60 from which sclerotomy points 350, 352 are derived and marked can be made before any incisions are made, or they can be determined right before they are needed.

Likewise, the first and second double-armed sutures 616 can be prepared prior to the making of any incisions 618, 620. The prosthesis 100, 200 could be provided for the surgical methods of the invention with pre-cannulated pre-loaded sutures and surgical needles already coupled thereto as described herein. In addition, it will be appreciated that some surgeons may not physically mark the eye 500 with the above-described marks at all, but the sclerotomies will still be marked in a virtual sense by making the sclerotomies in substantially the same places based on the same considerations as disclosed herein, even if by the experienced eye of a surgeon.

Finally, those of skill in the art will appreciate that there is a certain tolerable margin of error with regard to the precision with which those points are determined, and the measurements made to determine them. Thus, the word “substantially” is often used herein to modify such determinations to account for that margin. This is also true with regard to centration of the optic centroid, the central aperture centroid and the optical or visual axes. This is a process that is also typically done by eye, However, the process has been described herein with regard to the ideal centration of the IOL, with the recognition that the ideal is not easily achievable by eye, but is the ideal goal of the surgeon, nevertheless. Thus, substantially centered is defined to be within an acceptable margin of error for a successful outcome.

What is claimed is:

1. A method of surgically implanting an intraocular lens (IOL) into an eye using a capsular prosthesis to support posterior chamber fixation, the IOL including an optic with haptics coupled thereto, said method comprising:
providing a capsular prosthesis comprising a sheet of substantially biocompatible and/or bioinert material, the sheet further including:
an anterior and posterior face separated by a thickness,
three or more vertices, each one of the vertices being uniquely associated with a suture aperture located proximally with its point; and
a center aperture located centrally with the vertices and being dimensionally configured to permit supportive optical capture of the IOL without substantial impairment of optic functionality;
surgically implanting the prosthesis into the eye, said implanting including:
inserting the prosthesis into the eye through a primary incision;
securing the prosthesis to the sclera of the eye with at least two transscleral sutures to establish at least three points of contact between the sclera and the at least three apertures of the prosthesis; and
configuring the at least two transscleral sutures to support the sheet within a desired plane located within the posterior chamber, the plane containing a predetermined surgical axis passing through the center aperture, the plane being approximately perpendicular to, and the center aperture of the sheet being functionally centered with, the predetermined axis of the eye;
inserting the IOL through a primary incision; and
optically capturing the IOL on the prosthesis so that a center of the optic is approximately centered with the aperture and the predetermined axis of the eye.

2. The method of claim 1, wherein:
a first one of the at least two transscleral sutures is secured to a first set of one or more of the at least three suture apertures by looping the first transscleral suture through each of the first set of the suture apertures, and
a second one of the at least two transscleral sutures is secured to a second set of one or more of the at least three suture apertures by looping the second transscleral suture through each of the second set of the suture apertures.

3. The method of claim 2, wherein:
the sheet of the prosthesis is substantially rectangular,
the first set of the suture apertures includes two of the suture apertures each located proximally with a different one of two vertices located at a first end of the sheet, and
the second set of the suture apertures includes two of the suture apertures each located proximally with a different of two vertices at a second end of the sheet.

4. The method of claim 2, wherein:
the sheet of the prosthesis is substantially triangular in geometry,
the first set of the suture apertures includes an aperture located at the apex of the triangular sheet, and
the second set of the suture apertures includes two of the suture apertures each located proximally with a different one of the two vertices defining the base of the triangular sheet.

5. The method of claim 2, wherein each of the first and second looped transscleral sutures has two paired ends, and said securing the prosthesis to the sclera further includes:
for each one of the paired ends of the first looped transscleral suture, identifying a sclerotomy point that is proximal to a first predetermined point along the predetermined surgical axis, and
for each one of the paired ends of the second looped transscleral suture, identifying a sclerectomy point made proximally with a second predetermined point located along the predetermined surgical axis and approximately 180 degrees from the first predetermined point.

6. The method of claim 5, wherein the first and second predetermined points are about 4 mm posterior to the surgical limbus of the eye.

7. The method of claim 6, wherein the sclerotomy points identified for each of the paired ends of the first and second transscleral sutures are on opposite sides of the predetermined surgical axis approximately 3 mm from the first and second predetermined points respectively.

8. The method of claim 7, wherein the primary incision is made at a first predetermined incision point along the predetermined surgical axis.

9. The method of claim 7, wherein the primary incision is made at a first incision point along an axis that is approximately perpendicular to the predetermined surgical axis.

10. The method of claim 9, further including loading the first and second looped transscleral sutures through the first and second sets of apertures respectively prior to surgery, each of the paired ends being coupled to a surgical needle.

11. The method of claim 10, wherein said securing the prosthesis to the sclera further includes:
for each of the paired ends of the first transscleral suture, making a sclerotomy from outside of the eye substantially at the identified sclerotomy point using a hollow needle until a proximal end of the hollow needle becomes visible behind the pupil of the eye,
inserting the surgical needle into the eye through the primary incision; and
docking the inserted needle into the proximal end of the hollow needle and loading the inserted needle until the inserted needle emerges outside of a distal end of the hollow needle remaining outside of the eye.

12. The method of claim 11, wherein said securing the prosthesis to the sclera further includes:
for each of the paired ends of the second transscleral suture, making a sclerotomy from outside of the eye substantially at the identified sclerotomy point using a hollow needle until a proximal end of the hollow needle becomes visible behind the pupil of the eye,
inserting the surgical needle of the paired end into the eye through the primary incision; and
docking the inserted needle into the proximal end of the hollow needle and loading the inserted needle until the inserted needle emerges outside of a distal end of the hollow needle remaining outside of the eye.

13. The method of claim 12, wherein after removing the needles from the paired ends, the method further comprises pulling both paired ends of the first suture to pull the prosthesis within the eye through the primary incision.

14. The method of claim 12, further comprising pulling the paired ends of both sutures to suspend the prosthesis within the eye and so that it approximately occupies the desired plane.

15. The method of claim 10, the method further including inserting the IOL into the eye through the primary incision using a standard lens insertion cartridge.

16. The method of claim 10, further including manipulating the optic with a surgical instrument so that its longitudinal edges are in contact with one of the faces of the prosthesis, so that the optic is substantially centered with the center aperture of the prosthesis, and the haptics of the IOL are captured within vertex features defined by the center aperture to resist further displacement.

17. The method of claim 10, wherein the predetermined surgical axis is determined to match the axis of astigmatism of the eye to facilitate easier placement of the IOL, and the IOL is a single-piece toric lens.

18. The method of claim 5, wherein said securing the prosthesis to the sclera includes subjecting each of the paired ends of the first and second looped transscleral sutures to heat cautery to make thickened flanges to secure the looped transscleral sutures within the sclera of the eye.

19. The method of claim 5, wherein said securing the prosthesis to the sclera further includes tying the paired ends of the first and second looped transscleral sutures to secure the looped transscleral sutures within the sclera of the eye.
